(19) United States
(12) Patent Application Publication
(10) Pub. No.: US 2018/0244987 A1
(43) Pub. Date: Aug. 30, 2018

(54) PARTICLE COMPRISING AT LEAST ONE FERRIMAGNETIC OR FERROMAGNETIC IRON OXIDE NANOPARTICLE ASSOCIATED WITH AT LEAST ONE COMPOUND FOR MEDICAL OR COSMETIC USE

(71) Applicant: NANOBACTERIE, Paris (FR)

(72) Inventors: Edouard ALPHANDÉRY, Paris (FR); Imène CHEBBI, Palaiseau (FR)

(21) Appl. No.: 15/753,593

(22) PCT Filed: Oct. 21, 2016
(86) PCT No.: PCT/FR2016/000173

§ 371 Date: Feb. 20, 2018

(30) Foreign Application Priority Data
Oct. 21, 2015 (FR) 15/02228

(51) Int. Cl. C09K 11/06; A61K 9/51; G01N 33/52; C12P 3/00

(52) U.S. Cl. CPC C09K 11/06; A61K 9/5115; C09K 2211/1018; C12P 3/00; G01N 33/52

(57) ABSTRACT

A particle probe including at least one ferrimagnetic or ferromagnetic iron oxide nanoparticle associated with at least one compound, in which the at least one compound dissociates from the at least one ferrimagnetic or ferromagnetic iron oxide nanoparticle and/or chemically modifies itself, following a physicochemical disturbance applied on the particle probe.

### FIELD OF THE INVENTION

The field of the invention relates to particles comprising at least one ferrimagnetic or ferromagnetic iron oxide nanoparticle associated with at least one compound and their medical or cosmetic use.

### STATE OF THE ART

The performance of some new probes relies on the local detection of a parameter such as temperature. In order to achieve this, fluorescent thermometers have been developed containing iron oxide nanoparticles associated with a fluorophore using methods that usually avoid the quenching of luminescence by iron oxide, (S. K. Mandal et al, Langmuir, Vol. 21, P. 4175 (2005)). However, these nanoparticles are superparamagnetic and are therefore weakly magnetic. They have an unstable magnetic moment at physiological or ambient temperature.

New modes of drug delivery have also been proposed by associating drugs with nanoparticles, including magnetosomes, to improve the efficacy of drugs, (J-B. Sun et al, Cancer Letters, Vol. 258, P. 109 (2007)). However, in this case the drug can't be released in a controlled manner.

### DESCRIPTION OF THE INVENTION

An object of the present invention is the use, in particular in vitro, of a particle comprising: at least one ferrimagnetic iron oxide nanoparticle associated with at least one compound, in which the compound dissociates from the iron oxide nanoparticle and/or chemically modifies itself following a physicochemical disturbance applied on the particle, as a probe.

More particularly, an object of the present invention is the use, in particular in vitro, of a particle comprising:

at least one ferrimagnetic or ferromagnetic iron oxide nanoparticle associated with at least one compound, wherein the nanoparticle is preferably not bound to the compound by at least one nucleic acid or at least one amino acid, in which the compound dissociates from the iron oxide nanoparticle and/or chemically modifies itself, where the dissociation and/or chemical modification of the compound does not preferentially lead to its destruction, following a physicochemical disturbance applied on the particle, where the physicochemical disturbance preferentially induces a modification of condition of the nanoparticle, as a probe.

The particle according to the invention can comprise at least 1, 10, 10², 10³, 10⁴, 10⁵, 10⁶, 10⁹, 10²⁰, or 10⁴⁰ iron oxide ferrimagnetic or ferromagnetic nanoparticle(s) and/or at least 1, 10, 10², 10³, 10⁴, 10⁵, 10⁶, 10⁹, 10²⁰, or 10⁴⁰ compound(s).

The particle according to the invention, when associated with a luminescent substance, differs in its use from the luminescent nanoparticle probes usually described. Indeed, its original mode of use relies on the detection of a variation of luminescence due to the dissociation and/or chemical modification of a luminescent substance associated with the particle, following a physicochemical disturbance applied on the particle.

In an embodiment, the luminescence is associated with at least one property of luminescence that varies by less than 10⁻¹⁰, 10⁻⁵, 10⁻¹, 1, 10, 25, 50, 75, or 90% whereas the variation of luminescence is associated with at least one property of luminescence that varies by more than 10⁻¹⁰, 10⁻⁵, 10⁻¹, 1, 10, 25, 50, 75, or 90%, where this percentage of variation can be measured as a function of time or between at least two different spatial positions of the particle.

The particle according to the invention, when it is associated with a pharmaceutical compound, also differs in its use from the nanoparticles associated with drugs. Indeed, its original mode of use relies on the dissociation of the pharmaceutical compound from the particle, following a physicochemical disturbance applied on the particle.

The in vitro use of the particle can correspond to any use of this particle taking place outside of a human or animal, without any contact with a human or animal or without any administration in the organism of a human or of an animal.

The in vitro use of the particle according to the invention can correspond to any use of this particle occurring before the administration or the introduction of this particle into an organism or part of this organism or after the exit of this particle from the organism or part of this organism consecutive to the administration or the introduction of this particle into the organism or part of this organism. Preferably the organism is a human or animal organism. Preferably, the part of this organism is a biological substance, DNA, RNA, a protein, an enzyme, a lipid, a tissue, a cell, an organelle, the blood, or a tumor.

In an embodiment of the invention, the in vitro use of the particle can correspond to a medical treatment, e.g. of diagnostic or therapy, which is localized in part of an organism and does not affect the whole organism, or affects less than 90, 50, 25, 10, 5, 1, or 0.1% of this organism or of the cells of this organism. The in vitro use of the particle can correspond to a medical treatment that affects at least one cell, preferably a prokaryotic or eukaryotic cell, a tissue, an organ or at least one biological substance that is different from a cell, tissue, organ or biological substance present in an organism in its normal condition, such as a cell, tissue, or cancer organ or such as a bacterium or a virus.

In one embodiment, the use of the particle according to the invention as a probe makes it possible to detect, measure a parameter such as temperature, pH, a chemical environment, or radiation. In some cases, it can then be used as a diagnostic tool.

In another embodiment, the use of the particle according to the invention as a probe makes it possible to modify a parameter, such as the condition of a cell or a physiological condition. In some cases, it can then be used as a treatment tool.

The particle according to the invention is an assembly of atoms, molecules, in the solid, liquid, or gaseous state, preferably in the solid state.

In one embodiment of the invention, the particle is not or does not comprise crystalline liquid or dendrimer.

The compound is a luminescent substance and/or a pharmaceutical compound.

The luminescent substance is a substance able to emit light when it is or has been exposed to light radiation. It can be phosphorescent or fluorescent.

The pharmaceutical compound is a compound which is used either for medical applications, in particular for diagnosis or treatment, in particular as medical device or drug, either for cosmetic or biological applications, in particular for research purposes.

In one embodiment of the invention, NANOF designates a ferrimagnetic or ferromagnetic iron oxide nanoparticle, also sometimes equivalently designated as nanoparticle, the nanoparticle or the iron oxide nanoparticle.

A ferrimagnetic or ferromagnetic iron oxide nanoparticle according to the invention, designated here by NANOF, possesses at least one of the following properties: (i), a core that is usually crystallized, where this crystallinity can be characterized by the presence of at least one crystalline plane, preferably at least five crystalline planes, more preferably at least ten crystalline planes, and whose composition is iron oxide, preferably at least partly consisting of maghemite, magnetite or a mixture of maghemite and magnetite, most preferably a composition mainly consisting of magnetite, (ii) a coating, also sometimes called membrane, either of biological origin, composed for example of lipids and proteins, or of non-biological origin, where this coating can enable a transfer of energy or particle between the compound and the NANOF, stabilize the NANOF, make the NANOF biocompatible, promote NANOF cellular internalization, couple NANOF to different types of substances including the luminescent substance, a substance of medical interest, of low natural abundance, (iii), a behavior usually ferrimagnetic or ferromagnetic, in particular at physiological temperatures, (iv), a single magnetic domain called mono-domain, (v), a possible chain arrangement preventing or limiting aggregation and possibly promoting cellular internalization, (vi), a size measured in the presence or absence of a coating between 1 nm and 1 μm, between 10 nm and 200 nm, between 20 nm and 150 nm, (vii), the possible presence of a charge, preferably a surface charge, where this charge may be negative, especially when the nanoparticles are coated with negatively charged lipids, where this charge can vary as a function of pH, of the existence or not of a coating, and can promote cellular internalization, especially when it is positive or negative, (viii), at least one metal atom, such as an iron atom, and at least one other atom, such as an oxygen atom, whose magnetic moments are preferably antiparallel, leading to properties that can be ferrimagnetic, or (ix), at least one metal atom, such as an iron atom, and at least one other atom, such as another iron atom, whose magnetic moments are preferably parallel, leading to properties that can be ferromagnetic.

In one embodiment of the invention, the iron oxide nanoparticle is either entirely ferrimagnetic or entirely ferromagnetic, but it may happen in some cases that the alignment of the magnetic moments differs between the surface and the core of the nanoparticle, for example when one considers a nanoparticle of iron oxide whose heart contains iron and whose surface is iron oxide.

In one embodiment of the invention, the iron oxide nanoparticle is ferrimagnetic or ferromagnetic below the Curie temperature or the melting temperature of the nanoparticle or above the blocking temperature of the nanoparticle or at a pH of 7, 6, 5, 4, 8, 9 or 10, or when the anisotropic energy of the nanoparticle is higher than the thermal energy of the nanoparticle, which can lead to a thermally stable magnetic moment of the nanoparticle.

In some embodiments of the invention, it may happen that the nanoparticle loses its ferromagnetic or ferrimagnetic properties, for example when it is dissolved in vivo, when it is internalized in cells or lysosomes, especially at pH values which differ significantly from the physiological pH.

The ferrimagnetic or ferromagnetic behavior of NANOF results in a thermally stable magnetic moment, especially at physiological temperatures and in a coercivity, remanent magnetization, saturation magnetization, magnetocrystalline anisotropy, capacity to produce heat under the application of an alternating magnetic field, which is(are) non-zero, preferentially high. The NANOF can thus have a coercivity larger than 1, 10, 100, 200, 500, 1000, or 10,000 Oe, a ratio between remanent magnetization and saturation magnetization larger than 0.01, 0.1, 0.2, or 0.5, a saturation magnetization larger than 0.1, 1, 10, 20, 50, 60, 80, 90, or 100 emu per gram of NANOF, or a specific absorption rate (SAR) larger than 0.01, 0.1, 1, 10, 100, 200, 500, or 1000 Watt per gram of NANOF. The coercivity, the ratio between remanent magnetization and saturation magnetization, or saturation magnetization, are preferably measured at a temperature above 0.1, 1, 10, 50, 100, 200, 400, or 800 K. The SAR is preferably measured at a concentration in NANOF larger than 0.01, 0.1, 1, 5, 10, 15, 20, 50, 100, or 200 mg per ml, preferably by applying an alternating magnetic field of strength larger or lower than 0.1, 1, 2, 5, 10, 20, 50, 100, 1000, or 10000 mT and/or of a frequency larger or lower than 1, 10, or 100 Hz, 1, 10, 100, 1000, or 10000 KHz. These magnetic properties are generally better than those of superparamagnetic iron oxide nanoparticles, called SPION, which are commonly used for applications, including medical applications. SPION are smaller in size than NANOF and possess a magnetic moment usually thermally unstable at physiological temperatures. The ferrimagnetic or ferromagnetic behavior of the NANOF may also result in at least one of the following properties: (i), an interaction between the magnetic moment of the NANOF and a dipole, magnetic moment, of the luminescent substance resulting in a quenching of the luminescence of this luminescent substance, usually more efficient than for SPION, (ii), a coupling between the magnetic moment of the NANOF and a radiation, preferably a magnetic field, more preferably an alternating magnetic field, making it possible to induce a variation in luminescence, preferentially more important than for SPION, (iii), a movement in the organism of the compound or of the particle that can more easily be induced by the application of a magnetic field, preferably a magnetic field gradient, more preferably an alternating magnetic field gradient, or (iv), a cellular internalization of the compound or particle favored by the application of a magnetic field, preferably an alternating magnetic field, which can in particular enable the compound or the particle to measure intracellularly a variation of the environment of the compound or the particle or the properties of a radiation to which it is exposed.

It is equivalent to say that the NANOF is associated with the compound than to say that the compound is associated with the NANOF. In this case, the compound possesses at least one of the following properties. It can be: (i), bound to the NANOF, (ii), in interaction with the NANOF, (iii), integrated in the NANOF, notably during the preparation of the particle, (iv), integrated in the NANOF, (v), derived from the NANOF, in particular when it detaches from the NANOF during the use of the particle, (vi), incorporated into or adsorbed onto the crystallized core of the NANOF, (vii), incorporated in the NANOF or adsorbed onto the coating covering the NANOF, (viii), in the environment of the NANOF, (ix), in direct contact with the NANOF, that is to say in particular without any substance being between the compound and the NANOF, or (x), in indirect contact with the NANOF, that is to say in particular with at least one substance, a polymer, or a solvent, which is positioned between the NANOF and the compound.

In one embodiment of the invention, the substance, polymer, or solvent, positioned between the NANOF and the compound, is not synthesized by a living organism, is not a protein, a lipid, an enzyme, DNA, a strand of DNA, RNA, an RNA strand, one or more nucleic acid(s), one or more amino acid(s), is not an active biological substance, or is not streptavidin or biotin. In this case, the NANOF may not be bound to the compound by at least one such substance, polymer or solvent, positioned between the NANOF and the compound.

In one embodiment of the invention, the NANOF is not bound to the compound by at least 1, 10, 10², 10³, 10⁶, 10¹⁰, 10²⁰, or 10⁴⁰ amino acid(s), nucleic acid(s), DNA strand(s), RNA strand(s), protein(s), lipid(s), or enzyme(s).

In one embodiment of the invention, the substance, polymer, or solvent, positioned between the NANOF and the compound, may be at least one atom, such as carbon, or complex of atoms, issued from a living organism, from a protein, a lipid, DNA, or RNA, where this atom or complex of atoms has preferably been denatured by chemical and/or thermal treatment.

In one embodiment of the invention, the substance, polymer, or solvent, positioned between the NANOF and the compound, represents less than 90, 50, 25, 10, 5, 1, or 0.1% of the mass of the NANOF, where this percentage may correspond to the mass of the substance, polymer, or solvent, divided by the mass of the NANOF.

In one embodiment of the invention, the substance, the polymer, or the solvent, positioned between the NANOF and the compound, is not denatured or destroyed, following the physicochemical disturbance applied on the particle, or is not responsible for the dissociation of the compound from the NANOF and/or for the chemical modification of the compound, following the physicochemical disturbance applied on the particle. This denaturation or destruction may be associated with a loss or decrease in activity of the substance, polymer, or solvent, such as a loss or decrease in activity of a protein, a lipid, an enzyme, a transformation of double-stranded DNA into single-stranded DNA, or a loss of at least one amino acid or at least one nucleic acid by a complex of amino acids or nucleic acids.

In one embodiment, the NANOF is responsible for the dissociation of the compound from the NANOF and/or for the chemical modification of the compound, following the physicochemical disturbance applied on the particle.

In one embodiment of the invention, the compound is in direct contact with the NANOF when the distance between the compound and the NANOF is lower than 10 μm, 1 μm, 100 nm, 10 nm, or 1 nm.

The environment of the NANOF can be a liquid, solid or gaseous medium or at least 1, 10, 10², 10³, 10⁶, 10¹⁰, or 10⁴⁰ substance(s), preferentially one or more substance(s) different from the compound, which surround(s) or include(s) the particle over a distance measured from the center or the outer surface of the particle preferably lower than 1 m, 1 dm, 1 cm, 1 mm, 1 μm, 100 nm, or 10 nm.

The environment of the NANOF can be a liquid, solid or gaseous medium or at least 1, 10, 10², 10³, 10⁶, 10¹⁰, or 10⁴⁰ substance(s), preferentially one or more substance(s) different from the compound, which surround(s) or include(s) the particle over a distance measured from the center or the outer surface of the particle preferably larger than 1 m, 1 dm, 1 cm, 1 mm, 1 μm, 100 nm, or 10 nm.

The environment of the NANOF can be a liquid, solid or gaseous medium or at least 1, 10, 10², 10³, 10⁶, 10¹⁰, or 10⁴⁰ substance(s), preferentially one or more substance(s) different from the compound, which surround(s) or include(s) the particle over a distance measured from the center or outer surface of the particle preferably lower than or larger than the diameter of the earth, or than 1000 km, or than 1 km, or than 1 m, or than 1 dm, or than 1 cm, or than 1 mm, or than 1 μm, or than 100 nm, or than 10 nm.

According to the invention, the compound is said to dissociate from the NANOF when it separates from the NANOF to end up in the environment of the NANOF.

In one embodiment of the invention, the particle is inactivated or is in an inactivated state when no physicochemical disturbance is applied on it.

In another embodiment, the particle is activated or is in an activated state when a physicochemical disturbance is applied on it.

The probe according to the invention may contain the particle in an activated and/or inactivated state.

A NANOF and/or compound and/or magnetosome, modified or not, may, according to the invention, be in an activated or inactivated state.

A physicochemical disturbance can be applied using a human-made device, such as an MM, or naturally by the natural environment of the particle, such as a radioactive environment.

The dissociation of the compound from the NANOF can be characterized by a dissociation constant, K_{D}, which can be equal to the ratio between the concentration of the compound bound to the NANOF, measured when the particle is activated, divided by the same concentration, measured when the particle is inactivated. Preferably, the constant K_{D} according to the invention can be lower than 1, 0.9, 0.8, 0.7, 0.6, 0.5, 0.4, 0.3, 0.2, 0.1, 0.05, 0.02, 0.1, 0.05, 0.01, 10⁻³, 10⁻⁴, 10⁻⁵, 10⁻⁶, 10⁻⁷, 10⁻⁸, or 10⁻⁹. According to this definition, the smaller the K_{D} is, the larger the dissociation of the compound from NANOF is.

A measurement of the bond or interaction strength between the compound and the NANOF can be given by the value of the dissociation constant, K_{D}. The larger is the K_{D} the stronger the bonder strength between the compound and the NANOF is.

The compound is said to modify itself chemically when its composition and/or structure change(s), inducing a modification of some of its properties, in particular luminescent and/or pharmacological properties.

In one embodiment of the invention, the dissociation and/or chemical modification of the compound do(es) not result in its destruction or denaturation or preserve(s) the compound. In this case, the dissociation and/or chemical modification of the compound do(es) not preferentially trigger its denaturation or total or partial destruction, or the loss of at least 1, 10, 10², 10⁵, 10¹⁰, or 10²⁰ atoms, or the loss of at least 0.1, 1, 5, 10, 25, 50, or 90% of its weight, or the loss of at least one of its properties such as the loss of a property of luminescence or a pharmacological property, or the decrease of more than 1, 5, 10, 25, 50, 90% of its luminescence intensity, or the loss of its therapeutic or diagnostic activity. A physicochemical disturbance is preferably a disturbance caused by radiation and/or by variation of the environment of the particle, of the NANOF or the compound.

The physicochemical disturbance applied on the iron oxide NANOF can induce a modification of condition of the NANOF, preferentially selectively without inducing such a modification of condition in the compound. This may be the case when the NANOF is exposed to a physicochemical disturbance that induces a modification of condition such as a movement, or a temperature increase of the NANOF. Physicochemical disturbance may induce a larger modification of condition, such as a larger temperature variation, a more important movement, or a more important property modification, in the NANOF than in the compound. In order to demonstrate this difference in modification of condition, the physicochemical disturbance can be applied on the NANOF alone and on the compound alone and the change of one of the properties of the NANOF alone can be measured and be more important than the change of this same property for the compound alone, following the application of this disturbance.

The physicochemical disturbance is preferably a disturbance which does not irreversibly modify the properties of the compound or NANOF, preferentially the luminescent or therapeutic properties of the compound. Such a disturbance, leading to the denaturation of the compound or NANOF, consisting in an increase in temperature, beyond the melting or denaturing temperature of the compound or NANOF, is for example preferably avoided. It is preferred that the physicochemical disturbance is sufficiently moderate so that the compound maintains at least partly its properties, preferentially of luminescence or of therapeutic substance, following the application of this disturbance.

A physicochemical disturbance is preferably a sufficiently moderate disturbance, such as a moderate increase in temperature of the particle, preferably lower than 300, 200, 100, 50, 20, 10, or 1° C., which may result in a mass loss of the particle of less than 90, 50, 25, 10, 5, 1, or 0.1%.

A radiation may be an undulating or particulate radiation, a sound radiation, an ionizing radiation, an X-ray radiation, an electromagnetic radiation, a radiation due to an electric, magnetic or electromagnetic field, or a radiation caused by neutrons, protons, electrons, positron, alpha particle, beta, gamma, neutrinos or muons.

The environment of the particle may be a liquid, solid or gaseous medium or at least 1, 10, 10², 10³, 10⁴, 10⁵, 10⁶, 10⁹, 10²⁰, or 10⁴⁰ substance(s) surrounding or including the particle over a distance measured from the center or outer surface of the particle, preferably lower than 10 000 km, 1000 km, 100 km, 10 km, 1 km, 1 m, 1 dm, 1 cm, 1 mm, 1 μm, 100 nm, or 10 nm.

The environment of the particle according to the invention may be a liquid, solid or gaseous medium or at least 1, 10, 10², 10³, 10⁴, 10⁵, 10⁶, 10⁹, 10²⁰, or 10⁴⁰ substance(s) surrounding or including the particle over a distance measured from the center or outer surface of the particle, preferably larger than 10 000 km, 1 000 km, 100 km, 10 km, 1 km, 1 m, 1 dm, 1 cm, 1 mm, 1 μm, 100 nm, or 10 nm.

The environment of the particle according to the invention may be a liquid, solid or gaseous medium or at least 1, 10, 10², 10³, 10⁴, 10⁵, 10⁶, 10⁹, 10²⁰, or 10⁴⁰ substance(s) surrounding or including the particle over a distance measured from the center or outer surface of the particle preferably larger or lower than the earth's diameter, or than 10 000 km, or than 1000 km, or than 100 km, or than 10 km, or than 1 km, or than 1 m, or than 1 dm, or than 1 cm, or than 1 mm, or than 1 μm, or than 100 nm, or than 10 nm.

The environment of the compound according to the invention may be a liquid, solid or gaseous medium or at least 1, 10, 10², 10³, 10⁴, 10⁵, 10⁶, 10⁹, 10²⁰, or 10⁴⁰ substance(s) surrounding or including the compound over a distance measured from the center or outer surface of the compound preferably larger or lower than the diameter of the earth's diameter, or than 10 000 km, or than 1000 km, or than 100 km, or than 10 km, or than 1 km, or than 1 m, or than 1 dm, or than 1 cm, or than 1 mm, or than 1 μm, or than 100 nm, or than 10 nm.

The environment of the particle according to the invention may include the particle, the compound and/or the NANOF.

A substance of the environment of the particle, compound or NANOF according to the invention may be an atom, a molecule, a polymer, a chemical or biological substance, preferentially a substance different from the compound or NANOF, DNA, RNA, a protein, a lipid, an enzyme, or a nucleic or amino acid contained in this environment.

The environment of the particle, the compound or the NANOF according to the invention may be a biological environment, that is to say an environment comprising at least one biological substance such as a cell, an organelle, DNA, RNA, a protein, a lipid, an amino acid, or a nucleic acid.

The variation of the environment of the particle, of the NANOF or of the compound according to the invention may be a pH variation, preferably lower or larger than 14, 13, 12, 11, 10, 9, 8, 7, 6, 5, 4, 3, 2, 1, or 0.1 pH units, a variation in temperature, preferably lower or larger than 500, 400, 300, 200, 100, 50, 25, 10, 5, 1, or 0.1° C., a variation in redox potential, preferably lower or larger than 1000, 100, 10, 5, 2, 1, 0.1, or 0.01 V, a variation in viscosity, preferentially in dynamic viscosity, preferentially lower or larger than 10²⁰, 10¹⁰, 10⁵, 10³, 10², 10, 10⁻¹, 10⁻², 10⁻³, 10⁻⁶, 10⁻⁹, or 10⁻²⁰ Pa·s, or a variation in chemical composition of this environment. The variation in the chemical composition of this environment may correspond to the variation in concentration of at least one substance in this environment and may be preferably lower or larger than 100, 10, 1, 10⁻¹, 10⁻², 10⁻³, 10⁻⁶, or 10⁻⁹M.

In the present description, we notably distinguish the case where the particle is inactivated and does not undergo any physicochemical disturbance from the case where the particle is activated and undergoes such disturbance.

The conditions under which the particle functions according to the invention correspond to the conditions under which the particle switches from an inactivated state to an activated state or vice versa from an activated state to an inactivated state.

For certain modes of uses of the particle according to the invention, the compound is in stronger interaction with the NANOF when the particle is inactivated than when the particle is activated. This can particularly be the case when the use of the particle involves the dissociation of the compound from the NANOF.

For other modes of use of the particle according to the invention, the interaction between the NANOF and the compound may not vary or even increase when the particle switches from the inactivated state to the activated state. This may especially be the case when the use of the particle involves a chemical modification of the compound.

A diagram is shown in FIG. 1 which illustrates the use of the particle according to the invention involving either a dissociation of the compound from the NANOF or a chemical modification of the compound.

In one embodiment of the invention, the ferrimagnetic or ferromagnetic iron oxide nanoparticle (NANOF) is synthesized by a magnetotactic bacterium and does not comprise any carbonaceous material originating from the bacterium.

In one embodiment of the invention, the NANOF is synthesized by a living organism, preferably a bacterium, more preferably a magnetotactic bacterium.

Following an embodiment of the invention, NANOF are nanoparticles synthesized by magnetotactic bacteria, especially selected from the group consisting of Magnetospirdlum magneticum AMB-1, coccus MC-1, vibrios MV-1, MV-2 et MV-4, Magnetospirdlum magneticum MS-1, Magnetospirdlum gryphiswaldense MSR-1, spirillum, Magnetospirillum magneticum MGT-1 and Desulfovibrio magneticus RS-1.

A NANOF synthesized by a magnetotactic bacterium is a magnetosome, especially as defined by D. A. Bazylinski et al, Nature Reviews Microbiology, V. 2, P. 217 (2004).

A NANOF according to the invention can be synthesized chemically or by another living organism than a magnetotactic bacterium and have at least one property identical or similar to that of a magnetosome, modified or not.

A magnetosome according to the invention may have at least one property identical or similar to that of a NANOF.

In one embodiment of the invention, the magnetosomes are in the form of a chain comprising between 2 and 1000 magnetosomes, between 2 and 500 magnetosomes, between 2 and 250 magnetosomes, between 2 and 100 magnetosomes, between 2 and 75 magnetosomes, typically between 4 and 20 magnetosomes, preferentially maintained arranged in chains by material, of biological origin (lipids and proteins for example) or non-biological.

The magnetosomes belonging to these chains usually have crystallographic directions, easy magnetization axes, or field lines, which are aligned in the direction of the chain elongation. This alignment makes it possible, among other things, to stabilize the magnetic moment of the magnetosomes. Consequently, magnetosome chains possess a significant magnetic anisotropy. When several chains of magnetosomes containing at least X magnetosomes interact, it results in the formation of a longer chain of magnetosomes containing at least X+1 magnetosomes.

The length of a magnetosome chain according to the invention may be lower or larger than 1 m, 1 dm, 1 cm, 1 mm, 100 μm, 10 μm, 1 μm, 600 nm, 300 nm. Because of their chain arrangement, the magnetosomes do not aggregate and have a stable magnetic moment that can couple itself to the magnetic field, thus promoting the luminescence variation of the said probe. The chain arrangement also promotes cellular internalization of magnetosomes, which is enhanced by the application of an alternating magnetic field.

In one embodiment of the invention, in order to determine whether the NANOF or magnetosomes are in the form of chains or not, a few microliters of a suspension of NANOF are deposited on a support, usually a carbonaceous grid, and the NANOF are analyzed under electron microscope. NANOF are considered to be arranged in a chain when they have crystallographic axes or directions, in particular at least 1, 2, 5, 10, 20, 30, 50 directions aligned in the direction of the chain elongation. Such alignment may mean that the angle between the crystallographic direction of a NANOF and the direction of elongation of the chain is less than 90, 80, 50, 40, 20, 10, or 5°. NANOF that do not have this property are not arranged in chains. In one embodiment of the invention, the magnetotactic bacteria can be cultured in a medium, such as that described in patent application WO2011/061259 incorporated by reference.

In one embodiment of the invention, the culture medium of the magnetotactic bacteria contains at least one source of iron, such as a solution of iron quinate, iron citrate, iron chloride, iron malate, or ferrous sulfate, especially at concentrations between 0.02 μM and 2 μM, 0.02 μM and 200 mM, 0.2 μM and 20 mM, 2 μM and 2 mM, or between 20 μM and 200 μM and one or more other additives usually added at concentrations between 0.01 μM and 1 M, 0.01 μM and 100 mM, 0.01 μM and 10 mM, 0.01 μM and 1 μM, 0.1 μM and 100 μM, or between 1 μM and 10 μM. These additives are preferably chosen among transition metals, chelating agents, luminescent substances, substances/isotopes of low frequency/natural abundance, substances having the properties of several of the above mentioned substances. Bacteria or these additives may be introduced into the culture medium during at least one of the different growth phases of the magnetotactic bacteria, such as the latency phase, the acceleration phase, the exponential phase, the deceleration phase or the stationary phase and/or the bacteria can be cultured in the presence of a low concentration of oxygen, lower than 1000 bar, 100 bar, 10 bar, 1 bar, 200 mbar, or 20 mbar and/or the pH or another parameter of the growth medium of the magnetotactic bacteria is maintained at a constant or almost constant value during the growth of the magnetotactic bacteria, for example by adding an acidic or basic solution preferably comprising a certain concentration of these additives. Moreover, these additives and bacteria growth conditions are preferably chosen from those which make it possible to avoid toxicity, which confer good magnetic and/or luminescent properties of the said probe, which allow the additives to be incorporated into the magnetotactic bacteria and/or in magnetosomes, which produce an increase in size and/or length of the magnetosome chains, which stimulate the growth of magnetotactic bacteria, which make it possible to achieve a sufficiently high production yield of magnetosomes, especially larger than 0.1 mg, 1 mg, 10 mg, 100 mg, 1 g, 10 g of magnetosomes per liter of culture, which make it possible to obtain a narrow magnetosome size distribution, in particular lower than 60 nm, 30 nm, 15 nm, 10 nm, 5 nm, or 1 nm.

In one embodiment of the invention, the association of the luminescent substance to the magnetosomes may be obtained (or not) by genetic manipulation of the magnetotactic bacteria.

In one embodiment of the invention, the luminescent substance is preferably not a fluorescent protein, such as a green or yellow fluorescent protein, a fluorescent enzyme, a fluorescent amino acid, a fluorescent nucleic acid, DNA or strand of fluorescent DNA, or RNA or strand of fluorescent RNA.

Another object of the invention is a method for producing the particle comprising the cultivation of magnetotactic bacteria in the presence of a luminescent substance followed by the extraction of the magnetosomes from these bacteria, their purification and their characterization.

Another object of the invention is a method for producing the particle comprising the cultivation of the magnetotactic bacteria in the absence of the luminescent substance followed by the extraction of the magnetosomes from these bacteria, their purification, the association of the luminescent substance to the magnetosomes and their characterization.

In one embodiment of the invention, the NANOF is a modified magnetosome.

A modified magnetosome is preferably a magnetosome which does not comprise, essentially not, or in small amounts, organic or carbonaceous material, consisting in particular of lipids, proteins, DNA, RNA, or enzymes, which can in particular surround the mineral core of the magnetosomes.

In one embodiment of the invention, a mineral does not comprise carbon or less than 75%, 50%, 25%, 10%, 5%, 2%, 1%, or 0.5% of carbon.

In one embodiment of the invention, a modified magnetosome according to the invention comprises less than 100%, 90%, 80%, 70%, 60%, 50%, 40%, 30%, 20%, 10%, 5%, 1%, or 0.1% of carbonaceous material, where this percentage of carbonaceous material can be defined as being the ratio between the number of carbonaceous atoms comprised in a modified magnetosome and the total number of atoms comprised in a modified magnetosome.

A modified magnetosome is preferably a nanocrystal consisting mainly of a mineral core and possibly of a coating that does not come from the magnetotactic bacterium. A modified magnetosome may have at least one property in common with an unmodified magnetosome, such as a chain arrangement.

A preparation of modified magnetosomes according to the invention is preferably pyrogen-free, and does not comprise, or does not essentially comprise, or comprises in small quantity endotoxin or lipopolysaccharide.

In one embodiment of the invention, a preparation of modified magnetosomes comprises less than 10⁶, 10⁵, 10⁴, 10³, 10², 10, 1, 10⁻¹, 10⁻², 10⁻³, 10⁻⁴, or 10⁻⁵ EU (endotoxin unit) per mL per mg, per mg, or per cm³ of modified magnetosome or of preparation of modified magnetosomes.

In one embodiment of the invention, the largest dimension of the particle is preferably comprised between 1 nm and 10 μm or 10 nm and 1 μm.

In one embodiment of the invention, the largest dimension of the particle is preferably lower than 100 μm, 50 μm, 10 μm, 1 μm, 500 nm, 250 nm, 100 nm or 50 nm.

In one embodiment, the particle according to the invention further comprises a substance selected from the group consisting of an isotope of low frequency or natural abundance, a transition metal, and a substance of medical interest.

Advantageously, an isotope of low frequency or natural abundance may be associated with the particle to facilitate the detection of the particle in a medium comprising a low quantity or none of this isotope (for example iron 54, 57 or 59 which are minimally present in nature).

Advantageously also, a transition metal may be associated with the particle in order to improve its magnetic properties, in particular to facilitate the detection of its luminescence variation.

Advantageously also, a substance of medical interest such as a targeting agent, a substance used during a medical operation, a therapy, a diagnosis, a surgery session, radiotherapy, hyperthermia, Mill, chemotherapy may be associated with the particle or compound, in particular to facilitate or improve its use or medical efficacy.

In one embodiment of the invention, the particle according to the invention may be encapsulated in a compartment, preferably a vesicle, most preferably a lipid vesicle such as a liposome, a giant vesicle or a nanogel, in particular in order to improve the efficacy or decrease the toxicity of the particle.

In one embodiment of the invention, the particle according to the invention is associated, bound or in interaction with a substance that does not belong to the particle. This can enable the detection of this substance or a condition of this substance. It may also make it possible to improve the medical, therapeutic, diagnostic, magnetic, luminescent and heating properties of the particle, or to reduce the risks of toxicity, in particular by preventing direct contact between the particle and the organism.

In one embodiment of the invention, ferrimagnetic or ferromagnetic iron oxide nanoparticle (NANOF) is synthesized chemically.

In one embodiment of the invention, the compound is associated with the NANOF by weak bonds. This situation is preferably encountered when the use of the particle according to the invention involves dissociation and/or chemical modification of the compound from/of the NANOF.

The weak bonds are preferably bonds allowing the dissociation of the compound from the particle under physicochemical disturbance where this disturbance is preferentially such that it does not modify significantly or it modifies in an insignificant manner at least one of the properties of the NANOF such as its size, size distribution, ferrimagnetic or ferromagnetic property, composition, or crystallographic structure.

In one embodiment of the invention, a significant modification in a property of the NANOF can be defined as: (i), an increase or decrease in size of the NANOF larger than 100 nm, 1 μm, or 10 μm, (ii), an increase or a decrease in the size distribution of the NANOF of more than 100 nm, 1 μm, or 10 μm, (iii), a modification in magnetic property due for example to the transition of the NANOF from a single domain magnetic nanoparticle to a multi domain magnetic nanoparticle, which may in particular lead to the loss of ferrimagnetic or ferromagnetic property of the NANOF, (iv), a modification in composition such as the transition from a nanoparticle composed of iron oxide to a nanoparticle only composed of iron or oxygen, or (v), a modification of the crystallographic structure such as the transition from a nanoparticle in a crystallized condition, which can be characterized by the presence of at least two crystalline planes in the nanoparticle, to a nanoparticle in an amorphous condition, which can be characterized by the absence of crystalline planes in the nanoparticle.

In one embodiment of the invention, a non-significant modification in a property of the NANOF may be defined as: (i), an increase or decrease in the size of the NANOF of less than 100 nm, 1 μm, or 10 μm, (ii), an increase or decrease in the size distribution of the NANOF of less than 100 nm, 1 μm, or 10 μm, (iii), an absence of modification in magnetic property due to, for example, the NANOF transition from a single magnetic domain nanoparticle to a multi magnetic domain nanoparticle, which can notably lead to the loss of ferrimagnetic or ferromagnetic property of the NANOF, (iv), an absence of composition modification such as the transition from an iron oxide composition to an iron or oxygen composition, or (v), an absence of modification of the crystallographic structure such as the absence of a transition from a nanoparticle in a crystallized condition, which can be characterized by the presence of at least two crystalline planes in the nanoparticle, to a nanoparticle in an amorphous condition, which can be characterized by the absence of crystalline planes in the nanoparticle.

Preferably, the weak bonds according to the invention are hydrogen bonds, bonds which are due to Van der Waals, London, Debye forces, intermolecular or inter-nanoparticle forces, or hydrophobic, hydrophilic, or electrostatic interactions.

In one embodiment of the invention, the weak bonds may be associated with the adsorption of the compound onto the NANOF.

In one embodiment of the invention, the weak bonds may be characterized by a dissociation constant, K_{D}, which is preferentially lower than 1, 0.9, 0.8, 0.7, 0.6, 0.5, 0.4, 0.3, 0.2, 0.1, 0.05, 0.02, 0.1, 0.05, 0.01, 10⁻³, 10⁻⁴, 10⁻⁵, 10⁻⁶, 10⁻⁷, 10⁻⁸, or 10⁻⁹.

In one embodiment of the invention, the bond between the compound and the NANOF is stronger when the particle is inactivated than when it is activated. This property can notably be observed when the use of the particle involves a dissociation of the compound from the NANOF.

In one embodiment of the invention, the compound is not bound to the NANOF by strong bonds, such as covalent, ionic bonds, or bonds that prevent dissociation of the compound from the NANOF. This property is preferentially observed when the use of the particle involves a dissociation of the compound from the NANOF and/or a chemical modification of the compound.

In one embodiment of the invention, the compound is associated with the NANOF by strong bonds. This situation is preferentially met when the use of the particle involves a chemical modification of the compound.

The strong bonds are preferably bonds preventing the dissociation of the compound from the NANOF under a physicochemical disturbance where this disturbance is preferentially such that it does not significantly modify at least one of the properties of the NANOF such as its size, size distribution, ferrimagnetic or ferromagnetic property, composition or crystallographic structure.

In one embodiment, the strong bonds are the bonds that are not weak.

Preferably, the strong bonds according to the invention are covalent, metallic or ionic bonds.

In one embodiment of the invention, the strong bonds may be characterized by a dissociation constant, K_{D}, which is preferentially larger than 1, 0.9, 0.8, 0.7, 0.6, 0.5, 0.4, 0.3, 0.2, 0.1, 0.05, 0.02, 0.1, 0.05, 0.01, 10⁻³, 10⁻⁴, 10⁻⁵, 10⁻⁶, 10⁻⁷, 10⁻⁸, or 10⁻⁹.

In one embodiment of the invention, the particle comprises a compound that is luminescent.

The luminescent substance according to the invention has at least one luminescent property.

A luminescent property is defined herein as an intensity, a luminescence wavelength, an intensity, an absorption wavelength, a luminescence lifetime, or a luminescence anisotropy, which is preferentially measurable. The luminescence property can be attributed to the luminescent substance or the particle.

In one embodiment of the invention, the luminescent substance possesses at least one luminescence property that varies, preferably under the operating conditions of the particle.

A luminescence variation is defined herein as a variation of at least one luminescence property. It can be attributed to the luminescent substance or particle and can be an increase or decrease, preferably an increase, of at least one luminescence property of the particle or luminescent sub stance.

A luminescence property may vary over time, depending on the spatial position of the particle or NANOF, during a variation of the particle or NANOF environment, following the application of the radiation, dissociation of the luminescent substance from the NANOF and/or chemical modification of the luminescent substance.

In one embodiment of the invention, the luminescence variation is a continuous increase in luminescence. It occurs preferentially over time, over a period of time preferably larger than 10⁻¹⁰, 10⁻⁹, 10⁻⁸, 10⁻⁷, 10⁻⁶, 10⁻⁵, 10⁻⁴, 10⁻³, 10⁻², 10⁻¹, 1, 2, 5, or 10 seconds, 1, 2, 5, 10, 30 or 45, minutes, 1, 2, 5, 10, or 17 hours, 1, 2, 5, 10, 15, or 30 days, 1, 2, 5, or 10 months, 1, 2, 5, 10, 25, 50, or 100 years. It is preferably not a succession of at least 1, 10, 10², 10³, 10⁶, 10⁹, 10²⁰, or 10⁴⁰ increase(s) and decrease(s) of luminescence that can occur in particular during random interactions of the luminescent substance.

In one embodiment of the invention, the variation of luminescence (ΔL) over time is characterized by a slope at the origin, ΔL/δt, where δt represents a duration originating from the beginning of the luminescence variation, which is preferably determined during the first seconds or minutes following the physicochemical disturbance applied on the particle.

A luminescence property or its variation can be determined around, at the surface or in the particle or NANOF over a distance measured from the center of the particle or NANOF preferably lower than 1 m, 1 dm, 1 cm, 1 mm, 1 μm, 100 nm, or 10 nm.

In one embodiment of the invention, the luminescence variation is determined within a region enabling luminescence variation detection, i.e. a region comprising a sufficient number of particles, preferably larger than 1, 10, 100, 10³, 10⁴, 10⁵, 10⁶, 10⁷, 10⁸, 10⁹, 10¹⁰, or 10¹¹ particles or particles per unit volume, in particular particles per m³, cm³, mm³, or μm³.

In one embodiment of the invention, the luminescent substance possesses at least one luminescence property that is measurable over time and/or as a function of the position of the particle or NANOF, or undergoes a variation of at least one of its properties that is measurable over time and/or as a function of the position of the particle or NANOF.

In another embodiment of the invention, certain parameters such as the measurement time, the size and mass of the luminescent substance and NANOF, the type of bond between the luminescent substance and NANOF, or the properties of the environment of the particle such as its viscosity, are optimized to allow the measurement of a luminescence variation of the luminescent substance.

In one embodiment of the invention, the luminescent substance possesses at least one of the following properties: (i), a small size or a small mass, preferably lower than one tenth, one hundredth, or one thousandth that of the NANOF, which can in particular make it possible to promote the dissociation of the luminescent substance from the NANOF and/or its diffusion in the environment of the particle, (ii), a negative charge, preferably lower than −1, −10, −20, or −50 mV, a positive charge, preferably larger than 1, 10, 20, or 50 mV, a neutral charge, or a charge opposite to that of the NANOF, which may especially favor the bonding between the luminescent substance and the NANOF, (iii), a bond between the luminescent substance and the NANOF enabling its dissociation from the NANOF under the operating conditions of the particle, (iv), a crystallinity revealed by the presence of at least two crystalline planes, or, (v), a non-zero absorption at wavelengths where the biological medium absorbs in small amount, or a non-zero absorption between 20 nm and 2 μm or between 600 and 1200 nm.

In one embodiment of the invention, the preferred luminescent substance is that which achieves the strongest quenching of luminescence intensity and/or the smallest luminescence lifetime when the particle is inactivated.

In one embodiment of the invention, the preferred luminescent substance is the one that enables the most important energy or particle transfer with the NANOF when the particle is inactivated or activated.

In one embodiment of the invention, the preferred luminescent substance is that which makes it possible to induce a luminescence variation which is: (i) as large as possible and/or measurable when the particle switches from an inactivated to an activated state, or (ii), measurable when the particle is exposed to low intensity, low energy radiation and/or to a variation of the environment of the particle of low amplitude, such as a temperature variation lower than 10⁶, 10⁵, 10⁴, 10³, 10², 10, 5, 1, 0.5, or 0.1° C. or a pH variation lower than 100, 50, 20, 14, 10, 8, 6, 4, 2, 1, 0.5, 0.1, or 0.01 pH units.

In one embodiment of the invention, at least one luminescence property of the luminescent substance associated with the iron oxide nanoparticle differs from that of the luminescent substance alone. This difference is more easily observable and measurable when the particle is inactivated or activated, preferentially inactivated, than when the particle switches from the inactivated to the activated state or from the activated to the inactivated state.

In one embodiment of the invention, the luminescence intensity of the luminescent substance associated with the iron oxide nanoparticle is quenched by the iron oxide nanoparticle compared with the luminescence intensity of the luminescent substance alone. This quenching is preferably observed and measurable when the particle is inactivated.

In one embodiment of the invention, the quenching of the luminescence intensity of the luminescent substance is determined by measuring the ratio I_{L}/I_{NL} between the luminescence intensity of the luminescent substance bound to the NANOF, I_{L}, and the luminescence intensity of the luminescent substance alone, not bound to the NANOF, I_{NL}. This ratio, in particular I_{L}, is preferably measured when the particle is inactivated.

In one embodiment of the invention, I_{L} et I_{NL} are measured under the same conditions, in particular with regard to the parameters of excitation and detection of the luminescence, as well as the concentration of the luminescent substance used during the measurements.

In one embodiment of the invention, the luminescence intensity of the luminescent substance associated with the NANOF is quenched when I_{L}/I_{NL} is lower than 1, preferentially lower than 0.9, 0.8, 0.7, 0.6, 0.5, 0.4, 0.3, 0.2, 0.1, 0.05, 0.02, 0.01, 0.005, 0.001, 10⁻⁴, 10⁻⁵, 10⁻⁶, 10⁻⁷, 10⁻⁸, or 10⁻⁹.

In one embodiment of the invention, the luminescence lifetime of the luminescent substance associated with the iron oxide nanoparticle is modified, quenched, or increased, preferably increased, by the iron oxide nanoparticle compared with the luminescence lifetime of the luminescent substance alone. In the case where the luminescence lifetime is increased by the iron oxide nanoparticle, it is an unusual situation, different from that usually encountered in the case of a Förster transfer, leading to a decreased life time.

In one embodiment of the invention, the variation in the luminescence lifetime of the luminescent substance is determined by measuring the ratio T_{A}/T_{L} between the luminescence lifetime of the luminescent substance bound to the NANOF, T_{A}, and the luminescence lifetime of the luminescent substance alone, not bound to the NANOF, T_{L}.

In one embodiment of the invention, T_{A} and T_{L} are measured under the same conditions, in particular with regard to the parameters of excitation and detection of the luminescence as well as the concentration of the luminescent substance used during the measurements.

In one embodiment of the invention, the luminescence lifetime of the luminescent substance bound to the NANOF is decreased, in particular by the presence of the NANOF, when T_{A}/T_{L} is lower than 1, preferentially lower than 0.9, 0.8, 0.7, 0.6, 0.5, 0.4, 0.3, 0.2, 0.1, 0.05, 0.02, 0.01, 0.005, 0.001, 10⁻⁴, 10⁻⁵, 10⁻⁶, 10⁻⁷, 10⁻⁸, or 10⁻⁹.

In one embodiment of the invention, the luminescence lifetime of the luminescent substance bound to the NANOF is increased, in particular by the presence of the NANOF, when T_{A}/T_{L} is larger than 1, preferentially larger than 2, 4, 8, 10, 25, 50, 100, 1000, 10⁴, 10⁵, 10⁶, 10⁷, 10⁸, or 10⁹.

In one embodiment of the invention, the variation of at least one luminescent property of the luminescent substance associated with the NANOF compared with the luminescent substance alone occurs when the luminescent substance possesses at least one of the following properties: i), an energy or particle transferred to the NANOF or received from the NANOF, this energy or particle exchange being possibly a Förster or Dexter transfer, (ii) a separating distance separating the luminescent substance from the NANOF preferably lower than 1 cm, 1 μm, 500 nm, 100 nm, 50 nm, 25 nm, 10 nm, or 5 nm, or a separating distance preferably lower than 100 times, 100 times, 50 times, 20 times, 10 times, twice, or 1 time the Förster distance, where the Förster distance is preferably between 0.1 and 50 nm, 0.5 and 20 nm, or 1 and 10 nm, (iii), the characteristics of a donor or acceptor in the terminology used to describe the Förster transfer, (iv), the ability to emit light at the wavelength(s) where NANOF absorbs light, (v), a lifetime or luminescence intensity measured when the luminescent substance is alone, which is sufficiently different from that of the luminescent substance bound to the NANOF for the variation of the lifetime or luminescence intensity to be measurable, or (vi), a lifetime of the luminescent substance alone larger than 0.5 ns, 1 ns, 5 ns, 10 ns, 100 ns, 1 μs, 10 μs, 100 μs, or 1 ms.

In one embodiment of the invention, the variation of at least one luminescence property of the luminescent substance associated with the NANOF compared with the luminescent substance alone occurs when the NANOF has at least one of the following properties: (i), a non-zero, preferentially significant, absorption, absorption coefficient, absorption index, or extinction coefficient(s), at the emission wavelength of the luminescent substance, more preferably significant between 10 and 5000 nm, 20 and 2000 nm, or 100 and 1500 nm, (ii), a material contained in its core and/or coating that absorbs at the emission wavelength(s) of the luminescent substance, (iii), a larger size than that of the SPION, larger than 0.1 nm, 1 nm, preferably larger than 10 nm, more preferably larger than 20 nm, which may in particular make it possible to increase its absorption, its absorption coefficient, its absorption index, or its extinction coefficient, which can(may) increase with the size of the NANOF, (iv), ferrimagnetic or ferromagnetic properties which may also increase the absorption, absorption coefficient, absorption index, or extinction coefficient of the NANOF and/or promote the coupling between dipoles and/or magnetic moments of NANOF and those of luminescent substances, or (v), a good crystallinity, which can in particular enable a transfer of energy, particles, preferentially electrons, between the NANOF and the luminescent substance.

In one embodiment of the invention, the quenching of the luminescence intensity and/or variation of the luminescence lifetime of the luminescent substance associated with the NANOF compared with the luminescent substance alone occurs when the luminescence intensity of the luminescent substance and the molar extinction coefficient of the NANOF as well as the recovery function associated with them is non-zero, preferably maximized.

In one embodiment of the invention, the quenching of the luminescence intensity and/or the variation of the luminescence lifetime of the luminescent substance associated with the NANOF compared with the luminescent substance alone is(are) due to the presence of oxygen or iron atoms in or at the surface of the NANOF.

In one embodiment of the invention, the quenching of the luminescence intensity and/or variation of the luminescence lifetime of the luminescent substance associated to the NANOF compared with the luminescent substance alone is(are) observed when the particle is activated or inactivated. On the other hand, it can be more pronounced when the particle is inactivated than when it is activated.

In one embodiment of the invention, the dissociation of the luminescent substance from the iron oxide nanoparticle and/or the chemical modification of this substance is related to a luminescence variation of the luminescent substance. This occurs preferentially when the particle switches from an inactivated to an activated state.

In one embodiment of the invention, the dissociation of the luminescent substance from the NANOF suppresses or decreases the quenching of the luminescence intensity, resulting in an increase in luminescence intensity.

In one embodiment of the invention, the dissociation of the luminescent substance from the iron oxide nanoparticle and/or chemical modification of the luminescent substance results in a luminescence variation of the luminescent substance which is due to: (i), a modification (beginning, increase, stopping, or decrease) of a transfer of energy or particle between the luminescent substance and the NANOF, (ii), a modification in the distance separating the luminescent substance and the NANOF, or (iii), a modification in the chemical composition or structure of the luminescent substance.

In one embodiment of the invention, the dissociation of the luminescent substance from the NANOF and/or chemical modification of the luminescent substance causing the luminescence variation is: (i) reversible by involving dissociation of the luminescent substance from the NANOF and/or its chemical modification followed by a return to the initial state of the particle obtained when the particle is inactivated, (ii), irreversible involving the dissociation of the luminescent substance from the NANOF and/or its chemical modification without a return of the luminescent substance to its initial state, (iii), total involving the entire luminescent substance, (iv), partial involving a part, a chemical group, an atom of the luminescent substance, (v), associated with a movement, a translation, an oscillation, or a vibration of the luminescent substance or NANOF, (vi), associated with a coupling between an electromagnetic field, the luminescent substance and NANOF, (vii), due to a variation of the environment of the particle, or (viii), followed by diffusion of the luminescent substance or a part of it into the particle environment.

In one embodiment of the invention, the dissociation of the luminescent substance from the NANOF and/or the chemical modification of this substance is related to a local luminescence variation, occurring when the luminescent substance has diffused at a distance measured from the center of the NANOF, preferably lower than 100 times, 10 times, or 1 time the size of the NANOF. This local variation in luminescence is usually measured for a measurement time lower than 100, 10, or 1 minute.

In one embodiment of the invention, at least one parameter related to the dissociation of the luminescent substance and/or the chemical modification of this substance such as the number of dissociated and/or diffusing luminescent substance, their speed, or time of diffusion and/or dissociation, can be measured. It can then be possible to establish a relation between ΔL, ΔL/δt and at least one of these parameters.

In one embodiment of the invention, the variation of the environment of the particle is related to a luminescence variation of the luminescent substance. This preferentially occurs when the particle switches from the inactivated to the activated state.

In one embodiment of the invention, the variation of the environment of the particle and/or the slope at the origin of this variation can be measured before, after, or during, preferably during, the use of the particle. It may then be possible to establish a relationship between ΔL, ΔL/δt, and the variation of this environment such as a variation of pH, temperature, redox potential, or chemical composition of this environment.

In one embodiment of the invention, the variation of the radiation to which the particle is exposed, according to the invention, such as a variation of the strength, of the energy, of the frequency, of an electromagnetic radiation such as a magnetic field (alternating or not), or ionizing radiation, is related to a luminescence variation of the luminescent substance. This preferentially occurs when the particle switches from the inactivated to the activated state.

In one embodiment of the invention, the variation of the radiation to which the particle is exposed, according to the invention, and/or the slope at the origin of this variation can be measured before, after, or during, preferably during, the use of the particle. It may then be possible to establish a relation between ΔL, ΔL/δt, and the variation of this radiation.

In one embodiment of the invention, the luminescence variation of the probe according to the invention occurs when the said probe comes into interaction, for example in contact or collision, with a substance, synthetic or non-synthetic. This may especially occur during the internalization of the said probe in a cell, in particular by the application of radiation, preferably an ionizing radiation or a magnetic field.

In one embodiment of the invention, the luminescence variation of the luminescent substance associated with the iron oxide nanoparticle is different from that of the luminescent substance alone.

In one embodiment of the invention, the luminescent substance possesses at least one luminescence property or undergoes a variation of at least one of its properties which is different from that of the luminescent substance alone, i.e. unbound or not associated with the particle according to the invention.

In one embodiment of the invention, the physicochemical disturbance applied on the particle according to the invention induces a variation of at least one luminescence property of the luminescent substance which is different from that induced by this same disturbance on the luminescent substance alone. For example, an increase in the temperature of the particle environment or the application of an alternating magnetic field to the particle may induce the dissociation of the luminescent substance from the NANOF and/or a chemical modification of the luminescent substance inducing a luminescence variation (increase or decrease) of the luminescent substance associated with the NANOF which is different from the luminescence variation of the luminescent substance alone, not bound to the NANOF, exposed to the same temperature increase or to the same alternating magnetic field.

In one embodiment of the invention, the luminescent substance associated with the particle is a luminophore, a chromophore or a fluorophore.

In one embodiment of the invention, the physicochemical disturbance applied on the particle induces an increase or decrease in luminescence which is 10, 10², 10³, 10⁶, 10¹⁰, or 10²⁰ times larger or lower when the luminescent substance is associated with the NANOF than when the luminescent substance is alone, not associated with the NANOF.

In one embodiment of the invention, the physicochemical disturbance applied on the luminescent substance associated with the NANOF induces an increase in luminescence whereas this same disturbance induces a decrease in luminescence when it is applied on the luminescent substance alone, not associated with the NANOF.

In one embodiment of the invention, the physicochemical disturbance applied on the luminescent substance associated with the NANOF induces a decrease in luminescence whereas this same disturbance induces an increase in luminescence when it is applied on the luminescent substance alone, not associated with the NANOF.

In one embodiment of the invention, the physicochemical disturbance applied on the luminescent substance associated with the NANOF induces a variation in luminescence, preferably at least 10⁻¹⁰, 10⁻⁵, 10⁻¹, 1, 10, 25, 50, 70, or 90%, whereas this same disturbance does not induce luminescence variation, preferentially a luminescence variation lower than 10⁻¹⁰, 10⁻⁵, 10⁻¹, 1, 10, 25, 50, 70, or 90%, when it is applied on the luminescent substance alone, not associated with the NANOF. In this case, the percentage of luminescence variation can be defined as being the ratio between the luminescence measured before and after the application of the disturbance.

In one embodiment of the invention, the luminescent substance comprises at least one luminescent chemical atom or group, a luminescent molecule, a luminescent material, or a luminescent monomer or polymer.

In one embodiment of the invention, the luminescent substance comprises or consists of at least one substance selected from the group consisting of:
luminescent ADN, luminescent ARN, Alexa fluor, Alexa fluor 350, 405, 430, 488, 500, 514, 532, 546, 548, 555, 568, 594, 610, 633, 647, 660, 669, 680, 700, 750 ou 790, ATTO, an ATTO 590, 594, 610, 611x, 620, 633, 635, 637, 647, 647N, 655, 655, 665, 680, 700, 725, 740, an Allophycocyanine (APC), a conjugated APC, a Cy, an APC-Cy7, an aminocoumarin, an allophycocyanin (APC), APC-Cy7, an orange acridine, a 7-AAD, an azurite, AmCyan1, AcGFP1, Azami-Green, AsRed2, aminocoumarin, allophycocyanin, ethidium bromide boron-dipyromethene BODIPY, BODIPY TMR, BODIPY 581/591, BODIPY TR, BODIPY-FL, BODIPY 630/650 ou 650/665, Ethidium bromide, a luminescent cell, calcein, coumarin, a cyanin Cy, Cy 2, Cy3, Cy3B, Cy 3.5, Cy 5, Cy 5.5 or Cy 7, Cy3.5, an organic dye, a blue chromomycin waterfall A3, a cerulean, a CyPet, a “DyLight Fluor”, DyLight 350, 405, 488, 549, 550, 594, 633, 649, 650, 680, 750, 755 or 800, DRAQ5, DAPI, DCFH, DHR, dKeima-Red, Dronpa-Green, DsRed monomer, DsRed2 (“RFP”), a luminescent enzyme, an EBFP, EBFP2, ECFP, Emerald, EYFP, E2-Crimson, a fluorescein, FluoProbe, a FluoProbe 594, 647H, 682, 752, 782, FluorX, FAM, Fluorescein FITC, a GFPuv, a hydroxycoumarin, a hoechst 33342 or 33258, HcRed1, a HyPer, hydroxycoumarin, Hex, interposing luminescent DNA, propidium iodine, Indo-1 Fluo-3, a J-Red, a Kusabira-Orange, Katushka (TurboFP635), a lucipherase, a luminescent lipid, a trivalent lanthanide Ln³⁺, yellow Lucifer, Lissamine Rhodamine B LDS 751, a lanthanide (Lanthane, Cerium, Praseodyme, Neodyme, Promethium, Samarium, Europium, Gadolinium, Terbium, Dysprosium, Holmium, Erbium, Thulium, Ytterbium, Lutecium), a megastokes, methoxycoumarin, mithramycin, a marker of cellular functions, a mCFP, mKeima-Red, mTFP1 (Teal), Midoriishi-Cyan, mCitrine, mBanana, mOrange, mOrange2, mKO, mStrawberry, mRFP1, mCherry, mKate2, mKate (TagFP635), mPlum, mRaspberry, mNeptune, Monochlorobimane, methoxycoumarin, s semiconductor nanocristal, NBD R-Phycoerythrin (PE), a luminescent protein, green, red, yellow fluorescent protein, P3, Pacific Blue, Pacific Orange, PE-Cy5, PE-Cy7, PerCP, PhiYFP, PhiYFP-m, red 613, r-phycoerythrin (PE), rhodamine red-x, rox, red 613, rhodamine, rhodamine B, SNARF, SYTOX, SYTOX Blue, SYTOX Green, SYTOX Orange, S65A, S65C, S65L, S65T, a luminescent substance contained in an antibody labeling kit such as a “Biotin Tag”, “Fluoro Tag”, “Immuno Probe” or “Mix-n-Stain”, a luminescent tissue, a Texas Red, a TOTO-3, TO-PRO-3, TruRed, TRITC X-Rhodamine, TOTO-1, TO-PRO-1, TO-PRO: Cyanine: Monomer, Thiazole Orange, TOTO-3, TO-PRO-3, T-Sapphire, TagBFP, TagCFP, TurboGFP, TagGFP, TagGFP2, Topaz, TurboYFP, TurboRFP, tdTomato DsRed-Express2, TagRFP, TurboFP602, TurboFP635, TRITC, Tamara, Texas Red, TruRed, Venus, YOYO-1 Y66H, Y66F, Y66W, YPet, ZsGreen1, ZsYellow1, and a substance derived from the aforementioned luminescent substances or having at least one atom, a molecule, a luminescent chemical group in common with the aforementioned luminescent substances.

In one embodiment of the invention, the particle according to the invention is associated with a luminescent substance, which possesses at least one chemical function selected from the group consisting of carboxylic acid, phosphoric, sulfonic acid, ester, amide, ketone, alcohol, phenol, thiol functional groups, amine, ether, sulfide, acid anhydride, acyl halide, amidine, nitrile, hydroperoxide, imine, aldehyde, peroxide, and an acid, basic, oxidized, reduced, neutral, or positively or negatively charged derivative of these functions.

In one embodiment of the invention, the luminescent substance has at least one chemical function that allows weak or strong binding with the iron oxide nanoparticle, such as a carboxylic acid function.

In one embodiment of the invention, the particle according to the invention is associated with a pharmaceutical compound.

In one embodiment of the invention, the particle according to the invention is associated with a pharmaceutical compound which has at least one chemical function selected from the group consisting of carboxylic acid, phosphoric, sulfonic ester, amide, ketone, alcohol, phenol, thiol, amine, ether, sulfide, acid anhydride, acyl halide, amidine, nitrile, hydroperoxide, imine, aldehyde, peroxide, and an acid, basic, oxidized, reduced, neutral, or positively charged or negatively derivative of these functions.

In one embodiment of the invention, the pharmaceutical compound has a chemical function that allows weak or strong binding to the iron oxide nanoparticle, such as a carboxylic acid function.

In one embodiment of the invention, the pharmaceutical compound is a therapeutic substance, a diagnostic, a contrast, an anti-cancer, antibacterial, an anti-inflammatory agent, a vaccine, anesthetic, analgesic, antibiotic, antidepressant, an antidiuretic, antihistamine, antihypertensive, antipyretic, antiviral, antitussive, anxiolytic, bronchodilator, diuretic, laxative, psychotropic, sedative, or vasopressor.

In one embodiment of the invention, the pharmaceutical compound contains an active ingredient and optionally an excipient.

In one embodiment of the invention, the pharmaceutical compound when associated with the particle has more efficacy and/or lower toxicity than when it is alone. This may be due to the fact that it can dissociate itself from the particle and be activated in a controlled manner, for example when it has reached the organism or the desired part of the organism such as a tumor or a tumor cell.

In one embodiment of the invention, the physicochemical disturbance applied on the particle leads to the dissociation of the pharmaceutical compound from the iron oxide nanoparticle.

In one embodiment, the dissociation of the pharmaceutical compound from the nanoparticle is controlled by the application of a physicochemical disturbance. This can in particular make it possible to improve the efficacy and/or to reduce the toxicity of a therapeutic treatment or a diagnosis.

In one embodiment of the invention, the physicochemical disturbance is applied on the particle by radiation, preferably an ionizing radiation or a magnetic field, more preferably an alternating magnetic field.

In one embodiment of the invention, the radiation is such that it significantly modifies NANOF, as defined in the invention.

In another embodiment of the invention, the radiation is a continuous magnetic field of strength preferably between 0.01 mT and 100 T, 0.1 mT and 10 T, or 1 mT and 1 T.

In one embodiment of the invention, the radiation is an alternating magnetic field of strength preferably between 0.01 mT and 100 T, 0.1 mT and 10 T, or 1 mT and 1 T and preferentially including frequency between 0.001 Hz and 1000 MHz, 0.01 Hz and 100 MHz, 0.1 Hz and 10 MHz, 1 Hz and 1 MHz, 10 Hz and 500 kHz, 100 Hz and 500 kHz, 1 kHz and 250 kHz, or 10 kHz and 200 kHz.

In one embodiment of the invention, the radiation is an ionizing radiation of energy larger than 10¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵ 10⁻⁴, 10⁻³, 10⁻², 10⁻¹, 1, 10, 10², 10³, 10⁴, 10⁵, 10⁶, 10⁷, 10⁸, 10⁹, 10¹⁰, 10¹¹, 10¹², 10¹³, 10¹⁴, or 10¹⁵ Gray (Gy).

In one embodiment of the invention, the radiation is an ionizing radiation of energy lower than 10¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵ 10⁻⁴, 10⁻³, 10⁻², 10⁻¹, 1, 10, 10², 10³, 10⁴, 10⁵, 10⁶, 10⁷, 10⁸, 10⁹, 10¹⁰, 10¹¹, 10¹², 10¹³, 10¹⁴, or 10¹⁵ Gray (Gy).

In one embodiment of the invention, the physicochemical disturbance applied on the particle according to the invention is due to or associated with a variation of the environment of the particle.

In one embodiment of the invention, the physicochemical disturbance applied on the particle or the variation of the environment of the particle according to the invention is a pH variation of less than 14, 13, 12, 11, 10, 9, 8, 7, 6, 5, 4, 3, 2, 1, 0.5, 0.2, 0.1, 0.01, 0.005, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³ pH units of that environment or at least one substance comprised in that environment.

In one embodiment of the invention, the physicochemical disturbance applied on the particle or the variation of the environment of the particle according to the invention is a pH variation of more than 14, 13, 12, 11, 10, 9, 8, 7, 6, 5, 4, 3, 2, 1, 0.5, 0.2, 0.1, 0.01, 0.005, 0.001, 10⁻⁴, 10⁻⁵, 10⁻⁶, 10⁻⁷, 10⁻⁸, 10⁻⁹, 10¹⁰, 10⁻¹¹, 10⁻¹², or 10⁻¹³ pH units of that environment or at least one substance comprised in that environment.

In one embodiment of the invention, the physicochemical disturbance applied on the particle or the variation of the environment of the particle according to the invention is a temperature variation of less than 10¹⁰, 10⁹, 10⁸, 10⁷, 10⁶, 10⁵, 10⁴, 10³, 500, 250, 150, 100, 50, 40, 20, 30, 10, 5, 1, 0.1, 0.01, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³° C. of this environment or at least one substance comprised in this environment.

In one embodiment of the invention, the physicochemical disturbance applied on the particle or the variation of the environment of the particle according to the invention is a temperature variation of more than 10¹⁰, 10⁹, 10⁸, 10⁷, 10⁶, 10⁵, 10⁴, 10³, 500, 250, 150, 100, 50, 40, 20, 30, 10, 5, 1, 0.1, 0.01, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³° C. of this environment or at least one substance comprised in this environment.

In one embodiment of the invention, the physicochemical disturbance applied on the particle or the variation of the environment of the particle according to the invention is a standard potential variation of less than 10³, 500, 100, 10, 5, 4, 3, 2, 1, 0.5, 0.4, 0.3, 0.2, 0.1, 0.01, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³ Volt of this environment or at least one substance comprised in this environment.

In one embodiment of the invention, the physicochemical disturbance applied on the particle or the variation of the environment of the particle according to the invention is a standard potential variation of less than 10³, 500, 100, 10, 5, 4, 3, 2, 1, 0.5, 0.4, 0.3, 0.2, 0.1, 0.01, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³ Volt of this environment or at least one substance comprised in this environment.

In one embodiment of the invention, the physicochemical disturbance applied on the particle or the variation of the environment of the particle according to the invention is a variation of the concentration of at least one substance of this environment of more of 10¹⁰, 10⁹, 10⁸, 10⁷, 10⁶, 10⁵, 10⁴, 10³, 500, 250, 150, 100, 50, 40, 20, 30, 10, 5, 1, 0.1, 0.01, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³ mole per liter, micromole per liter, nano-mole per liter, mole per milliliter, micromole per milliliter, nano-mole per milliliter, mole per cubic meter, mole per cubic decimeter, mole per cubic centimeter, or mole per cubic millimeter.

In one embodiment of the invention, the physicochemical disturbance applied on the particle or the variation of the environment of the particle according to the invention is a variation in the chemical composition of at least one substance of this environment of less than of 10¹⁰, 10⁹, 10⁸, 10⁷, 10⁶, 10⁵, 10⁴, 10³, 500, 250, 150, 100, 50, 40, 20, 30, 10, 5, 1, 0.1, 0.01, 0.001, 10⁻⁴, 10⁻⁵, 10⁻⁶, 10⁻⁷ 10⁻⁸, 10⁻⁹, 10⁻¹⁰, 10⁻¹¹, 10⁻¹², or 10⁻¹³ mole per liter, micromole per liter, nano-mole per liter, mole per milliliter, micromole per milliliter, nano-mole per milliliter, mole per cubic meter, mole per cubic decimeter, mole per cubic centimeter, or mole per cubic millimeter.

In one embodiment of the invention, the physicochemical disturbance applied on the particle or the variation of the environment of the particle according to the invention is a modification of at least 1, 2, 3, 4, 5, 10, 25, 50, 100, 500, 10³, 10⁴, 10⁵, 10⁶, 10⁷, 10⁸, 10⁹, or 10¹⁰ substance (s) in this environment where this modification may be a chemical or structural modification and/or the appearance or disappearance of substance(s) from that environment.

In one embodiment of the invention, the physicochemical disturbance applied on the particle or the variation of the environment of the particle according to the invention is the variation of chemical composition of less than 2, 3, 4, 5, 10, 25, 50, 100, 500, 10³, 10⁴, 10⁵, 10⁶, 10⁷, 10⁸, 10⁹, or 10¹⁰ substances in this environment where this variation may be a chemical or structural modification and/or the appearance or disappearance of substance(s) in that environment.

In one embodiment of the invention, the physicochemical disturbance applied on the particle induces a modification of the condition of the particle that can be defined as either: (i) an increase or decrease in pH of the particle of more or less than 14, 13, 12, 11, 10, 9, 8, 7, 6, 5, 4, 3, 2, 1, 0.5, 0.2, 0.1, 0.01, 0.005, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³ pH units, (ii), a temperature increase or decrease of the particle of more or less than 10¹⁰, 10⁹, 10⁸, 10⁷, 10⁶, 10⁵, 10⁴, 10³, 500, 250, 150, 100, 50, 40, 20, 30, 10, 5, 1, 0.1, 0.01, 0.001, 10⁻⁴, 10⁻⁵, 10⁻⁶, 10⁷, 10⁻⁸, 10⁻⁹, 10⁻¹⁰, 10⁻¹¹, 10⁻¹², or 10¹³° C., (iii), an increase or decrease in charge of the particle of more or less than 10³, 500, 100, 10, 5, 4, 3, 2, 1, 0.5, 0.4, 0.3, 0.2, 0.1, 0.01, 0.001, 10⁻⁴, 10⁻⁵, 10⁻⁶, 10⁻⁷, 10⁻⁸, 10⁻⁹, 10⁻¹⁰, 10⁻¹¹, 10⁻¹², or 10⁻¹³ Volt, or (iv), an increase or decrease in the number of atoms comprised in the particle of more or less than 10¹⁰⁰, 10⁵⁰, 10²⁰, 10¹⁰, 10³, 10, 5, or 1 atom(s).

The present invention also relates to an in vitro method for detecting luminescence variation, comprising a step of detecting luminescence variation of the particle according to the invention.

In one embodiment of the in vitro method for detecting luminescence variation according to the invention, the luminescence variation is measured during a time interval of less than one hour, one minute, or one second.

In another embodiment of the in vitro method for detecting luminescence variation according to the invention, the luminescence variation is measured locally around the particle, preferably at a distance measured from the center of the particle lower than 1 cm³, 1 mm³, or 0.1 mm³. In another embodiment of the in vitro method for detecting luminescence variation according to the invention, the luminescence variation is a continuous increase in luminescence of the luminescent substance, preferably for at least one second, one minute, or one hour.

In another embodiment of the in vitro method for detecting luminescence variation according to the invention, the dissociation of the luminescent substance from the NANOF is detected.

In another embodiment of the invention, the method for detecting the luminescence variation according to the invention is applied to detect at least one physiological parameter, preferably the temperature, the pH, the environment of the iron oxide nanoparticle.

In another embodiment, the method for detecting the luminescence variation according to the invention is applied to detect radiation as defined above.

In another embodiment of the in vitro method for detecting luminescence variation according to the invention, the luminescence intensity of the particle according to the invention is increased by applying a radiation as defined above.

In another embodiment of the in vitro method for detecting luminescence variation according to the invention, the luminescence intensity of the luminescent substance is increased by varying the environment of the particle.

The present invention also concerns a device comprising:
The particle according to the invention, wherein the compound is a luminescent substance, and
A system for detecting a variation in luminescence.

Preferably, the device according to the invention comprises a luminescent substance associated with the particle whose luminescence is spontaneous and a detection system making it possible to measure the luminescence variation of the luminescent substance associated with the particle.

Also preferably, the device according to the invention comprises a luminescent substance associated with the particle whose luminescence is induced by a first excitation source and detected by a detection system making it possible to measure the luminescence variation of the luminescent substance associated with the particle.

The first excitation source induces the luminescence of the luminescent substance associated with the particle. It can be a light source, monochromatic or polychromatic, operating continuously or by pulses. It preferably produces a single photon or multiphoton excitation and emits at a wavelength preferably between 300 nm and 1500 nm. It may be a laser, such as a crystalline laser, the Yag laser emitting at 1064 nm, a dye laser, a gas laser, a laser diode, a free electron laser or a fiber laser. It can also be a lamp, such as a halogen lamp. The detection system is a hardware or set of hardware for detecting the luminescence variation of the luminescent substance associated with the particle. It may comprise an apparatus for selecting the wavelength of the radiation emitted by the said probe such as a spectral analyzer of wavelength, a spectrometer, a photosensitive sensor whose resistance varies as a function of the intensity of the light radiation captured (photoelectric, photovoltaic or photoresist cells for example), a scintillator, a photomultiplier, a channeltron, a photographic sensor, a detector for dispersive energy analysis, an infrared radiation detector, a photodetector.

In one embodiment of the invention, the device according to the invention also comprises a second excitation source, such as a radiation source, preferably a source of ionizing radiation, or a magnetic field source, preferably an alternating magnetic field. This second excitation source can make it possible to induce a luminescence variation of the luminescent substance associated with the particle.

In another embodiment of the invention, the device according to the invention comprises an optical assembly containing at least one material for transporting a light signal, such as an optical fiber, a microscope, a lens, which may be used to transport light from an excitation source to the luminescent substance associated with the particle, to excite the luminescent substance, to collect the luminescence of the luminescent substance and/or to transport the luminescence of the luminescent substance from the luminescent material to the detection system.

In another embodiment of the invention, the device according to the invention comprises one or more detection system(s) used to detect at least one parameter of the particle according to the invention, the presence of a substance/isotope of low frequency/natural abundance, the properties of radiation such as frequency, intensity, energy of electromagnetic or ionizing radiation, a transition metal, or a substance of medical interest. The detection system according to the invention may contain a TIMS (solid source thermionisation mass spectrometer) or an ICP-MS (Inductively Coupled Plasma Mass Spectrometer), a radioactive isotope detector (also called a radioisotope) such as a nuclear radiation detecting instrument, a gas, semiconductor, inorganic scintillation, organic liquid scintillation or solid organic scintillation detector, a photomultiplier, a Geiger counter, a physiological parameter probe, such as a temperature or pH probe, or a magnetic field detection probe.

In one embodiment of the invention, the device according to the invention allows the particle according to the invention to switch from an inactivated to an activated state, where this activation can in particular result in the variation of at least one luminescence property of the luminescent substance.

In one embodiment of the invention, the activation of the said device is carried out when the particle according to the invention is activated.

In one embodiment of the invention, the activation of the device according to the invention and/or of the particle according to the invention results in a variation of at least one luminescence property of the particle which is measurable in the absence of a second excitation source and the device does not need to comprise a second excitation source. This situation can for example occur when the variation of the environment of the particle induces a variation of luminescence.

In one embodiment of the invention, the activation of the device according to the invention and/or of the particle according to the invention results in a variation of at least one luminescence property of the particle which can't be measured in the presence of a second excitation source and a second excitation source is then included in the device.

In one embodiment of the invention, the device according to the invention functions according to at least one of the following sequences: the luminescence of the luminescent substance associated with the particle (sequence 1), the luminescence variation of the luminescent substance associated with the particle (sequence 2), and the detection of this luminescence variation (sequence 3). Usually, these sequences are successive and the sequence 3 takes place almost simultaneously with the sequences 1 and/or 2 since it is desirable to measure the luminescence variation just after the excitation of the particle in order to avoid luminescence losses. The time separating sequence 3 from sequences 1 and/or 2 may be less than one minute, preferably less than one second, most preferably less than one hundredth of a second. It may furthermore happen that sequences 1 and 2 take place simultaneously, for example when radiation induces both the luminescence and the luminescence variation of the particle, or that sequence 1 takes place after sequence 2.

In one embodiment of the invention, the particle according to the invention and/or the device according to the invention can be used to measure the presence of the particle and its spatial distribution. In this case, radiation as defined above can be used to increase the luminescence of the particle and make it more easily detectable by the device according to the invention. Furthermore, an optical system comprising an optical fiber that is moved horizontally over a sample containing the particle according to the invention can be used to measure the spatial distribution of the particle. It may then be possible to establish a relationship between a spatial luminescence variation of the particle and the particle distribution in a given medium.

The present invention also relates to a pharmaceutical composition comprising at least the particle according to the invention, optionally combined with a pharmaceutically acceptable vehicle.

In a preferred embodiment of the pharmaceutical composition according to the invention, the compound is a pharmaceutical compound.

According to another aspect of the invention, the particle according to the invention is used as a drug, in particular for medical, veterinary or plant applications.

The present invention also relates to a method for treating a disease, including cancer, in an individual, wherein the individual is administered a therapeutically active amount of the particle according to the invention.

The present invention also relates to a diagnostic composition comprising at least the particle according to the invention.

In a preferred embodiment of the diagnostic composition according to the invention, the compound is a luminescent substance as defined above.

In one embodiment of the invention, the particle according to the invention is used as a diagnostic agent, or an intravital probe.

The present invention also relates to a diagnostic method in which the particle according to the invention is administered to an individual.

The present invention also relates to a medical device comprising at least the particle according to the invention, wherein the compound is preferably a pharmaceutical compound or the luminescent substance.

In one embodiment of the invention, the medical device according to the invention is used for medical, veterinary or plant applications, especially in the treatment of a disease such as cancer.

The present invention also relates to a cosmetic composition comprising at least the particle according to the invention, in which the compound is a cosmetic active ingredient, optionally associated with a cosmetically acceptable vehicle.

According to another aspect of the invention, the particle according to the invention is used as a cosmetic agent.

According to the invention, the particle may be a medical device, a drug, a cosmetic composition, a therapeutic substance, a contrast or diagnostic agent.

According to another aspect of the invention, the particle according to the invention comprising a luminescent compound is used as a probe to detect a luminescence variation or a variation of a property of the NANOF, the luminescent substance and/or the environment of the particle.

In some embodiments, the luminescent compound is the luminescent substance.

In one embodiment of the invention, the particle according to the invention comprising a luminescent compound is used as a probe to detect the dissociation of the luminescent substance from the NANOF and/or the chemical modification of the NANOF.

In one embodiment of the invention, the particle according to the invention comprising a luminescent compound is used as a probe for detecting radiation as defined in the invention.

In one embodiment of the invention, the probe according to the invention comprising the particle associated with the luminescent compound may be used in vivo, ex vivo or in vitro, in the context of medical or non-medical research, in particular a treatment, diagnosis and/or inside, at the surface or outside of a substance, preferably biological.

In one embodiment of the invention, the particle according to the invention comprising a luminescent compound is used as a probe to detect a particular physiological condition that is different from normal, preferably resulting from a disease or medical operation.

According to one embodiment of the invention, the particle according to the invention is used as a probe to detect a particular physiological condition that is different from that of a subject in its normal condition. This particular physiological condition may result from a disease, a medical operation, the exposure of that organism to a substance or radiation, a fever, a cancer, an immune system response. It can also be any condition reached by a subject during an operation or medical intervention, a treatment, a diagnosis, a surgical operation, a biopsy session, cytopuncture, endoscopy, MM, scanner, colonoscopy, scintigraphy, PET-SCAN, X-rays, chemotherapy, hyperthermia such as magnetic hyperthermia. To do this, the particle can be brought into interaction, in contact, in association with an organism or administered to this organism being in a particular physiological condition. This can induce a variation in the luminescence of the particle different from that observed when the body is in its normal condition and thus allow detection of this particular physiological condition. For example, the particle may be introduced into the tumor of a patient undergoing hyperthermia treatment and the measured luminescence variation may be different from that observed in the absence of treatment, thereby demonstrating the activation of the treatment.

In one embodiment of the invention, the particle according to the invention comprising a luminescent compound is used as a probe to detect the association, the marking, the targeting, the delivery of the particle, NANOF or the luminescent substance.

According to one embodiment of the invention, the particle according to the invention is used to detect the marking, the targeting, the delivery of a substance. To carry out the marking, the particle can be associated with this substance. To target a substance, the particle may be sent or guided to that substance, for example by attaching to the particle an antibody that specifically recognizes that substance or applying a magnetic field orientated in the direction of that substance. To deliver a substance, the particle may be associated with that substance and delivered to a given location. The labeling, targeting, delivery of this substance can then be observed from a characteristic luminescence variation occurring during labeling, targeting, delivery.

In one embodiment of the invention, the particle according to the invention comprising a luminescent compound is used as a probe to detect the mixture, the removal of the particle from a medium.

The present invention also relates to the use of the particle according to the invention as a probe for detecting the removal of certain substances from a material, organism, medium, especially in the context of a detoxification or for isolating and purifying these substances. To do this, the particle can be attached to these substances and the complexes thus formed can be removed from the material, organism, medium, using for example a magnetic field. This removal can be observed from the specific luminescence variation occurring during removal.

The present invention also relates to the use of the particle according to the invention as a probe for monitoring the mixing of certain substances with a material, organism, or medium. To do this, the said probe can be attached to these substances and the complexes thus formed can be mixed with a material, organism, or medium, using a magnetic field. This mixture can be observed from the specific luminescence variation occurring during mixing.

In one embodiment of the invention, the particle according to the invention comprising a luminescent compound is used as a probe to detect a particular condition which is different from a normal condition, such as a high level of pollution, radioactivity, toxicity.

According to one embodiment of the invention, the particle according to the invention is used as a probe to detect a particular condition which is different from a normal condition, for example a condition due to certain parameters such as a level of radioactivity, pollution, radiation, toxicity, which is(are) different, preferentially higher, than normal. To do this, the particle may be placed in a location having a different level of these parameters, preferably higher than normal. This can induce a variation in luminescence, which is different from that observed in a location having a normal level of these parameters and thus allow the detection of a different level of these parameters, preferably higher than normal.

The present invention also relates to the use of the particle according to the invention as a probe for detecting the efficacy of a transfection or magnetofection. In this case, DNA or RNA can be associated with the particle and transfected into cells in the presence or absence of a magnetic field. The efficacy of these mechanisms can be demonstrated from the specific luminescence variations occurring during transfection or magnetofection.

The present invention also relates to the use of the particle according to the invention as a probe for detecting the internalization of the particle, for example in a cell or an organelle where this internalization can be observed from a specific variation of luminescence occurring during this internalization.

The present invention also relates to the use of the particle according to the invention as a probe and/or to detect the dissociation of a substance from the particle and/or its diffusion following in particular the application of a radiation or the variation of the environment of the particle. The dissociation of this substance and/or its diffusion can be highlighted from the specific luminescence variation that it(they) induce(s).

In one embodiment of the invention, the particle according to the invention or the luminescent substance is subject to a luminescence variation that can be used to detect at least one of the following parameters: (i) a variation of the environment of the particle, NANOF or luminescent substance as defined in the invention, (ii), a radiation as defined in the invention, (iii), the dissociation of the compound from the NANOF and/or the diffusion of the compound, or (iv), the distribution of the particle. To enable this detection, a relation can be established between the luminescence variation and at least one of these parameters, either by using a known or determined relationship or by using at least one detection system making it possible to measure at least one of these parameters.

The present invention also relates to the particle comprising at least one ferrimagnetic or ferromagnetic iron oxide nanoparticle associated with at least one compound, in which the compound dissociates from the iron oxide nanoparticle and/or chemically modifies itself, following a physicochemical disturbance applied on the particle, and the iron oxide nanoparticle is synthesized by a bacterium, preferably a magnetotactic bacterium, and does not comprise organic or carbonaceous material originating from the bacterium. In this case, the particle may comprise mineral material originating from the bacterium.

In one embodiment of the invention, the particle does not comprise carbonaceous or organic material originating from a living organism or the bacterium when it comprises less than 90, 75, 50, 25, 15, 10, 5, 3, 1, 10⁻¹, 10⁻², 10⁻³, 10⁻⁴, 10⁻¹⁰, or 10⁻²⁰% of this carbonaceous or organic material, where this percentage may correspond to the mass of carbonaceous or organic material originating from a living organism or from the bacterium comprised in the particle divided by the total mass of the particle.

In one embodiment of the invention, the particle does not comprise carbonaceous or organic material originating from a living organism or the bacterium when it is pyrogen-free or when it comprises an endotoxin concentration lower than 10³, 1000, 500, 100, 50, 10, or 5 endotoxin units per mg of particle.

In one embodiment of the invention, the particle does not comprise any organic or carbonaceous material originating from the bacterium when it is not derived from a genetically modified bacterium, where the genetic modification is intended to yield the synthesis of the luminescent sub stance.

The present invention also relates to the particle comprising at least one ferrimagnetic or ferromagnetic iron oxide nanoparticle associated with at least one luminescent substance, in which the luminescent substance dissociates from the iron oxide nanoparticle and/or chemically modifies itself, following a physicochemical disturbance applied on the particle, and the luminescent substance has a size at least 100 times smaller than that of the nanoparticle, and the luminescent substance is in direct contact with the nanoparticle, and the particle does not comprise any carbonaceous or organic material originating from a living organism.

In one embodiment of the invention, the luminescent substance has a size at least 2, 5, 10, 10², 10³, 10⁴, 10⁵, or 10¹⁰ lower than the size of the nanoparticle, which can make it possible to bind or associate a large number of luminescent substances with the nanoparticle.

In one embodiment of the invention, the luminescent substance has a small size, preferably lower than 10 or 1 nm, 100, 10, 5, 1 or 0.1 Å, or a small molecular weight, preferentially lower than 10⁶, 10⁵, 10⁴, 10³, 10², or 10 g/mol, which can make it possible to bind or associate a large number of luminescent substances with the nanoparticle.

In one embodiment of the invention, the luminescent substance is bound to or associated with the nanoparticle by at least one substance whose size is lower than 10 or 1 nm, 100, 10, 5, 1 or 0.1 Å, or whose molecular weight is lower than 10⁶, 10⁵, 10⁴, 10³, 10², or 10 g/mol, which can make it possible to bind or associate a large number of luminescent substances with the nanoparticle.

In one embodiment of the invention, it is preferred to avoid the use of certain large structures, larger than 10 or 1 μm, 100, 10 or 1 nm, 100, 10, 5, 1 or 0.1 Å, such as biological substances, proteins, lipids, DNA, RNA, or large molecules such as dendrimers, to bind or associate the luminescent substance with the nanoparticle. This type of structure can on the one hand limit the number of luminescent substances that can be bound or associated with the nanoparticle and on the other hand create a significant distance, preferentially larger than 10 or 1 μm, 100 or 10 nm, between the luminescent substance and the nanoparticle, and thus interfere with the performance of the probe or the particle, including the quenching of the luminescence of the luminescent substance when the luminescent substance is close to the nanoparticle.

In one embodiment of the invention, the surface/volume ratio of the nanoparticle is sufficiently high to allow the association of the compound or luminescent substance, is preferably larger than 10⁻¹⁰, 10⁻⁵, 10⁻³, 10⁻¹, 0.5, or 0.9 nm⁻¹.

In another embodiment of the invention, the surface/volume ratio of the nanoparticle is not too high to prevent the nanoparticle from losing its ferrimagnetic or ferromagnetic properties, is preferably lower than 10³, 10², 10, 1, 0.1, or 0.01 nm⁻¹.

In one embodiment of the invention, at least 2, 5, 10, 10², 10³, 10⁴, 10⁵, or 10¹⁰ luminescent substances are associated with or bound to the nanoparticle or to each nanoparticle, preferably to each nanoparticle on average. In some cases, the number of luminescent substances associated with or bonded to each nanoparticle on average can be estimated by measuring the ratio between the number of luminescent substances not associated to nanoparticles comprised in a given volume, measured when the particle is activated or inactivated, preferentially when the particle is activated, divided by the number of nanoparticles not associated with the luminescent substance comprised in the same volume, measured when the particle is activated or not activated, preferably when the particle is activated.

In one embodiment of the invention, the particle comprising the luminescent substance associated with or bonded to the nanoparticle has a SAR, a coercivity, a ratio between remanent magnetization and saturation magnetization, or saturation magnetization which is (are) at least equal or equal to 10⁻⁴, 10⁻³, 10⁻², 10⁻¹, 1, 1.001, 1.01, 1.2, 1.5, 2, 5, 10, or 100 times the SAR, coercivity, ratio between remanent magnetization and saturation magnetization, or saturation magnetization of the nanoparticle, respectively.

In one embodiment of the invention, at least two of the following environments may be equivalent: the chemical environment, the particle environment, the NANOF environment, the compound environment, the environment of the probe, the natural environment of the particle, the radioactive environment, the biological environment.

### DESCRIPTION OF THE FIGURE

FIG. 1: Schematic FIGURE showing the fabrication and use of the particle. Under the effect of ionizing radiation or a modification in the environment of the particle, the compound dissociates from NANOF and/or is modified chemically.

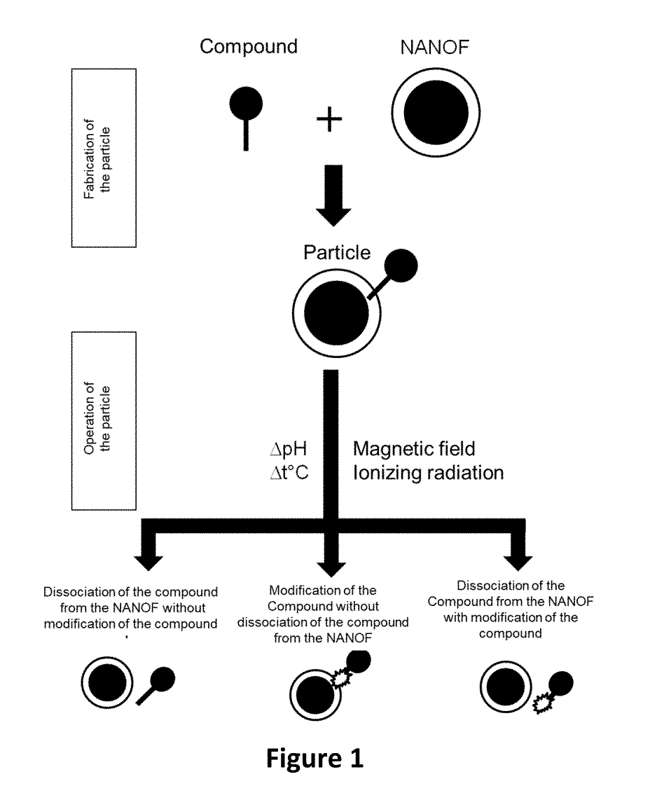

### EXPERIMENTAL EXAMPLES

### Signification of Abbreviations

In the description of the experimental examples, MC designates chains of magnetosomes extracted from non-luminescent magnetotactic bacteria grown in the absence of rhodamine B, MCR400 and MCR20 designate chains of magnetosomes extracted from magnetotactic bacteria, where the bacteria are synthesized in the presence of 400 μM of rhodamine B and 20 μM of rhodamine B. BNF-DiI and MC-DiI respectively designate plain BNF-Starch (Micromod 10-00-102) and magnetosomes in chains prepared in the presence of DiI (Sigma 42364; 1,1′-Dioctadecyl-3,3,3′,3′-tetramethyl-indocarbocyanine perchlorate). The supernatant of MCR400, BNF-DiI and MC-DiI is the liquid removed after having attracted the various iron oxide nanoparticles in suspensions using a 0.6 T Neodinium magnet.

### Example 1: Synthesis and Characteristics of MC and MCR400

The suspensions of MC and MCR400 were prepared according to the following experimental protocol. Magnetotactic bacteria Magnetospirillum magneticum AMB-1 were obtained from the ATCC (ATCC 700274) and were grown under micro-anaerobic conditions at 30° C. in a liquid culture medium slightly modified compared with the MSGM medium (ATCC Medium 1653). In one liter, this culture medium contains 0.68 g of monobasic potassium phosphate, 0.85 g of sodium succinate, 0.57 g of sodium tartrate, 0.083 g of sodium acetate, 225 μl of resazurin 0.2%, 0.17 g of sodium nitrate, 0.04 g of L-ascorbic acid, 2 ml of a 10 mM iron quinate solution, 10 ml of a solution of Woolf vitamins and 5 ml of a solution of Woolf minerals. To prepare the MC suspension, no rhodamine solution was added to the culture medium. To prepare the MCR400 suspension, 100 mL of a solution of 4 mM rhodamine B were introduced into the culture medium of the bacteria. To prepare the MCR20 suspension, 5 mL of a solution of 4 mM rhodamine B are introduced into the culture medium of the bacteria. The pH of the culture medium was adjusted to 6.85 using a 1M sodium hydroxide solution. The bacteria were collected during the stationary phase and the bacteria were concentrated using a column (mPES, 500 KDa) of tangential flow filtration with a flow rate of 950 mL/min and then washed 5 times for 30 minutes with a solution of saline phosphate buffer pH 7.4 (137 mM NaCl, 2.7 mM KCl, 10 mM Na₂HPO₄, 1.76 mM KH₂PO₄). The bacteria are collected by centrifugation at 4000 rpm for 1 hour, the supernatant is removed and the bacteria are resuspended in 50 mM Tris-HCl buffer solution at pH 7.4 and diluted to obtain an optical density of 5 at 600 nm. In order to extract the magnetosome chains, this suspension is sonicated at 5° C. for 60 minutes at 30 W (with a pulse of 2 sec and a pulse interval of 1 sec) to lyse the cell membranes, thus isolating the chains of magnetosomes from bacteria. After sonication, the magnetosome chains are magnetically isolated from cellular debris using a neodymium magnet. The supernatant containing the cellular debris is removed and the magnetosome chains are magnetically washed five times with a 50 mM Tris-HCl buffer solution at pH 7.4 and fifteen times with Millipore® water. They are finally re-suspended in Millipore® sterile water to obtain a suspension of MCR400, MCR20 or MC. The association of rhodamine B with MCR400 is revealed by the absorption spectrum of a MCR400 suspension at an iron concentration of 40 μg/mL. This spectrum displays a shoulder at 550 nm, absent in the absorption spectrum of MC and corresponds to the maximum absorption wavelength of rhodamine B. Moreover, the supernatant of the suspension of MCR400 absorbs in low amount at this wavelength. This confirms the fact that the absorption of rhodamine B in the suspension of MCR400 comes from rhodamine B associated with magnetosomes. This association can also be revealed by the fluorescence emission spectrum of MCR400. In fact, the suspension of MCR400, excited at 405 nm, has a maximum fluorescence intensity at 569 nm, a wavelength slightly lower than that corresponding to the maximum fluorescence intensity of rhodamine B, which is 576 nm. This difference could be explained by the association of rhodamine B with magnetosome chains which would slightly modify the emission wavelength of MCR400 compared with that of rhodamine B. Moreover, the infrared spectrum of MCR400 that we measured reveals the presence of deformation and vibration bands characteristic of MC (Amide I at 1650 cm⁻¹, amide II at 1530 cm⁻¹, lipopolysaccharide (LPS) or phospholipid at 1050 cm⁻¹ and at 1250 cm⁻¹, maghemite at 580 cm⁻¹) as well as those characteristic of rhodamine B (carboxylic acid at 1700 cm⁻¹, alkene at 1475 cm⁻¹ and 680 cm⁻¹, ether at 1250 cm⁻¹ and 1175 cm⁻¹, amine III at 1125 cm⁻¹). These results therefore seem to confirm that rhodamine B is either adsorbed at the surface of magnetosomes or complexed with magnetosomes, possibly due to the carboxylic acid function of rhodamine B. An experiment also made it possible to determine the number of rhodamine B molecules present on or in each magnetosome. For this, MCR400 were brought into the presence of hydrochloric acid at pH=2 in order to release rhodamine B from magnetosomes and transform the iron oxide into iron (III) and iron (II) ions. The fluorescence spectrum of MCR400 thus treated suggests that rhodamine B is well released from magnetosomes since the luminescence peak is at 582 nm (the emission wavelength of free rhodamine B in solution at pH 2) and not at 568 nm (the emission wavelength of rhodamine B associated with magnetosomes in MCR400). From the luminescence intensity of rhodamine B released from magnetosomes (˜0.56 a.u.), a rhodamine B concentration of 750 nmol/L is estimated from a calibration curve representing the luminescence variation of rhodamine B alone in solution at pH=2 as a function of the rhodamine B concentration. In 1 mL, there are therefore 4.6 10¹⁴ RhB molecules. Knowing the concentration of magnetosomes in suspension of ˜166 μg/mL and the mass of a magnetosome of 6.25 10⁻¹⁶ g, we deduce the number of magnetosomes in 1 mL as 26.5 10¹⁰ magnetosomes and the number of rhodamine B molecules associated with magnetosomes in MCR400 is estimated as ˜178±4. In other words, 178±4 molecules of rhodamine B are associated with each magnetosome on average.

We can conclude from this example that:

(i) We have presented a method for producing MCR400.
(ii) We have demonstrated the association of rhodamine B with magnetosomes in MCR400.
(iii) MCR400 are characterized by an absorption peak of wavelength similar to that of rhodamine B and by an emission peak at a different emission length than that of rhodamine B by about 7-10 nm.

### Example 2: Synthesis and Properties of MC-Dil and BNF-Dil

The suspensions of MC-DiI and BNF-DiI are prepared starting with a suspension of MC and BNF-starch, respectively, previously concentrated at 25 mg/mL in iron oxide by centrifugation at 14,000 rpm for 30 minutes at 5° C. A stock solution of DiI at 5 mg/mL is first prepared in previously distilled absolute ethanol. 5 mg in iron oxide of suspensions of MC and BNF-starch are then introduced into 200 μL of distilled absolute ethanol and then mixed with 2.5 mg of DiI coming from the stock solution. The mixture is sonicated at 30 W for 10 min (pulse 2 sec.), protected from light and at a temperature comprised between 5 and 10° C. A volume of 1 ml of sterile Millipore® water is added to the sonicated suspension and the sample is then left under magnetic stirring for 1 hour at room temperature and protected from light. Subsequently, this mixture is stored at 5° C. during 24 hours. The mixture is sonicated at 30 W for 10 min (pulse 2 sec.) protected from light at a temperature between 5 and 10° C. The suspensions of MC-DiI and BNF-DiI are concentrated by centrifugation at 14,000 rpm for 30 minutes at 5° C. and then washed five times with 0.5% ethanol and fifteen times with sterile Millipore® water. They are finally re-suspended in Millipore® sterile water to obtain suspensions of MC-DiI or BNF-DiI. The absorption spectra of DiI have two maximum at 560 nm and 520 nm. BNF-DiI show three absorption peaks at different wavelengths of 595 nm, 535 nm and 490 nm. The BNF-DiI supernate does not absorb at the absorption wavelengths of DiI. The emission spectra of DiI excited at 530 nm show two peaks of maximum intensity at 640 nm and 591 nm. As for the absorption spectra, a spectral shift is observed between the emission spectra of BNF-DiI excited at 530 nm, whose peaks of maximum intensity are at 670 nm and 600 nm, and those of the DiI. Moreover, the BNF-DiI supernate does not produce luminescence intensity at the emission wavelengths of DiI. The absorption and emission spectral shifts observed between BNF-DiI and DiI suggest a modification of DiI when it is associated with the BNF-Starch. The lack of absorption and emission of the BNF-DiI supernate suggests the absence of DiI in this supernate and the association of DiI with BNF in BNF-DiI.

MC-DiI show a relatively similar behavior to that of BNF-DiI with two absorption peaks at 570 nm and 520 nm, two emission peaks at 680 nm and 600 nm. As for BNF-DiI, the differences observed between the absorption and emission peaks of MC-DiI and those of DiI suggest a modification of DiI when it is associated with MC. The absence of absorption and emission of MC-DiI supernate suggests the association of DiI with MC in MC-DiI.

We can conclude from this example that:

(i) We presented a method for producing BNF-DiI and MC-DiI.
(ii) We highlighted the association of DiI with BNF and MC.
(iii) BNF-DiI and MC-DiI are characterized by absorption and emission peaks whose wavelengths are different from those of DiI.

### Example 3: Use of MCR400 as Temperature Probe

A solution of rhodamine B of concentration 5 μM and pH 7 contained in a volume of 800 μL is excited at 405 nm and heated at different temperatures for 30 minutes using a dry heating bath. When the temperature of this solution increases from 20° C. to 80° C., the wavelength corresponding to the maximum luminescence of rhodamine B remains relatively unchanged while the maximum luminescence intensity of rhodamine B decreases strongly by 73% in agreement with previously published results (Journal of Luminescence, Vol. 27, p. 455 (1982)). A suspension of MCR400 at a concentration of 250 μg/mL in iron oxide and pH 7 contained in a volume of 800 μl is excited at 405 nm and heated at different temperatures for 30 minutes using a dry hot-water heating bath. MCR400 excited at 405 nm produce maximum luminescence intensity at 569 nm. The behavior of the MCR400 is different from that of rhodamine B. Indeed, between 20° C. and 80° C., the maximum of the luminescence peak is observed at 569 nm and the luminescence intensity of the MCR400 increases sharply by 66%. The luminescence intensity of rhodamine B would initially be quenched when rhodamine B is associated with magnetosomes and would increase with the dissociation of rhodamine B from magnetosomes under the effect of a temperature increase.

The dissociation of rhodamine B from MCR400 by heating is confirmed by absorption measurements. Suspensions of MCR400 at concentration of 40 μg/mL in iron oxide are heated for a period of 0 to 240 minutes at pH 7 and the absorption of the supernate of MCR400 is measured at 550 nm. The variations of the absorption of the supernate of MCR400 are measured as a function of time and heating temperature. The longer the heating time of the MCR400 suspension is or the higher the heating temperature of the MCR400 suspension is, the larger the absorption of the supernate is. At 20° C., the absorption of the supernate of MCR400 increases from 0 to ˜0.004 in 240 minutes. At 60° C., the absorption of the supernate increases from 0.01 to ˜0.028 in 240 minutes. At 90° C., the absorption of the supernate increases from ˜0.022 to ˜0.044 in 240 minutes. When MCR400 are heated at different temperatures for 240 minutes, the concentration of rhodamine B in the supernate of the MCR400 suspension increases from 20 nM at 20° C. to 560 nM at 90° C.

We can conclude from this example that:

(i) It is possible to establish a relation between the luminescence intensity variation of MCR400 and the temperature variation of MCR400, and thus to use MCR400 as a temperature probe.
(ii) The luminescence intensity of MCR400 increases with increasing temperature, a behavior opposite to that of free rhodamine B in solution. This can in particular make it possible to distinguish between the presence of free rhodamine B and rhodamine B associated with magnetosomes.
(iii) The increase in MCR400 luminescence intensity with increasing temperature can be attributed to dissociation of rhodamine B from MCR400 when MCR400 are heated.
(iv) The amount of rhodamine B dissociated from the magnetosomes by heating can be evaluated by absorption measurements.

### Example 4: Use of MCR400 as a Probe to Detect Irradiation

Three suspensions are maintained at pH 7 and at a temperature of 25° C., containing either 400 μg/mL in iron oxide of MCR400, 125 μM of rhodamine B, or 400 μg/mL in iron oxide of MC mixed with 125 μM of rhodamine B. These suspensions are irradiated with a faxitron irradiator dosimeter (160 kV, 6.3 mA without filter, 67.5 Gy/min). The irradiation doses are between 0 and 1350 Gy. The luminescence, excited at 550 nm and measured at 578 nm, of suspensions containing rhodamine B alone or the supernate of rhodamine B mixed with MC do not vary or decrease when these suspensions are irradiated. In contrast, when the MCR400 suspension is irradiated, the luminescence of the supernate of this suspension, excited at 550 nm and measured at 576 nm, increases sharply from 250 a.u. in the absence of irradiation to 600-950 a.u. in the presence of an irradiation larger than 250 Gy. These results suggest the dissociation of rhodamine B from MCR400 under the effect of an irradiation. Moreover, the emission wavelength of the supernate of MCR400 and of free rhodamine B in solution remains unchanged at 576 nm, regardless of the irradiation dose received by MCR400 or rhodamine B. This suggests that free rhodamine B in solution or rhodamine B associated with magnetosomes has not been modified by irradiation.

We can conclude from this example that:

(i) It is possible to establish a relation between the variation of luminescence and the variation of irradiation of MCR400 and thus to use MCR400 as irradiation probe.
(ii) The luminescence intensity of the supernate of the MCR400 suspension increases with increasing irradiation, a behavior different from that of free rhodamine B in solution. This can in particular make it possible to distinguish between the presence of free rhodamine B and rhodamine B associated with magnetosomes.
(iii) The luminescence enhancement of MCR400 supernate with irradiation can be attributed to the dissociation of rhodamine B from MCR400 under irradiation.
(iv) The amount of rhodamine B dissociated from the magnetosomes by irradiation can be estimated by luminescence measurements.

### Example 5: Use of BNF-DiI as Temperature Probe

A suspension of DiI of concentration 5.4 μM and pH 7 is heated at various temperatures lying between 20 and 90° C. for 30 minutes using a dry heating bath. The maximum luminescence intensity of the DiI, excited at 530 nm, increases between 20 and 60° C. and then decreases between 60 and 100° C. When a suspension containing BNF-DiI mixed in water at a concentration of 80 μg/mL in iron oxide is heated at temperatures lying between 20 and 90° C. using a dry heating bath, the behavior is different from that observed with DiI alone. Indeed, the maximum luminescence intensity of BNF-DiI, excited at 530 nm, decreases between 20 and 60° C. and then remains almost stable between 60 and 100° C.

In order to determine if the luminescence variation of the BNF-DiI is due to the dissociation of DiI from the BNF, the maximum intensity of luminescence of the supernate of BNF-DiI and DiI alone, excited at 530 nm, were measured for suspensions containing BNF-DiI or DiI alone heated at different temperatures between 20 and 90° C. for a period of 0 to 30 minutes. The luminescence intensity of the supernate of BNF-DiI does not increase when BNF-DiI are heated between 20 and 90° C. for a period of 0 to 30 minutes. Given that when DiI is heated between 20 and 90° C. for a period of 0 to 30 minutes, the luminescence intensity of DiI does not significantly decrease, this suggests that the concentration of DiI in the supernate of the BNF-DiI suspension does not increase when the temperature increases and that DiI therefore does not dissociate from the BNF.

We can conclude from this example that:

(i) It is possible to establish a relation between the variation of the luminescence intensity of the BNF-DiI and the variation of temperature of the BNF-DiI and thus to use BNF-DiI as temperature probe.
(ii) The luminescence intensity of the supernate of BNF-DiI does not increase with increasing temperature, suggesting that DiI does not dissociate from BNF due to temperature increase. In this case, the decrease in luminescence of BNF-DiI with increasing temperature could be explained by a chemical modification of BNF-DiI.

### Example 6: Use of MCR400 as a pH Probe

MCR400 are tested as an in vivo pH probe on brains of rats. For that, 4 rats are used. Rat 1 is a healthy rat euthanized at the same time as rat 2. Rats 2 to 4 receive 5 μl of a suspension containing 3·10³ RG-2 cells implanted using a stereotactic helmet at coordinates 2 mm back, 2 mm lateral and 4 mm deep. Rat 2 is not treated and is euthanized 18 days after cell implantation. Rats 3 and 4 receive fourteen days following implantation of tumor cells at the site of cell implantation 10 μl of a suspension of MCR400 at a concentration of 668 μg/ml in iron oxide. Rats 3 and 4 are euthanized 2 hours and 4 days after the administration of the magnetosomes respectively. The brains are then extracted and cut into 3 mm thick layers. The luminescence of the slices is then produced using a pulsed laser diode of 40 MHz, 1 mW power, and 405 nm wavelength. The laser light is coupled to a 200 μm diameter optical fiber positioned 1.5 mm above the brain slices. The light emitted by the tissue is collected by a second optical fiber of diameter 365 μm located at 600 μm of the first fiber and positioned 1.5 mm above the brain slices. Luminescence is detected by a spectrometer (B. Leh et al., J. Biomed, Opt. (2012) 17 (10), 108001) enabling to measure the luminescence intensity of brain slices as a function of the wavelength of the emitted radiation. The two optical fibers can be moved laterally to different parts of the brain slices to identify the luminescent location.

For the different rats, the luminescence spectra of the healthy zones not containing MCR400 were measured, are very similar, displaying a luminescence band with a maximum intensity observed at about 500 nm. This band is due to the minority contribution of NADH luminescence at 450 nm and to the dominant contribution of flavoprotein at 525 nm. At 575 nm, a shoulder is observed which would come from the luminescence of lipo-pigments also called lipofuscin lipid debris. At 625 and 690 nm, the luminescence of protoporphyrin IX and of a derived molecule are observed. The spectra of the healthy zones were averaged thus making it possible to normalize all the measured luminescence spectra for all slices of rat brain.

For the different rats, the luminescence spectra of the tumor zones not containing MCR400 were also measured, are very similar, indicating the presence of peaks similar to those observed for the healthy are with lower intensities.

For the rat 3 euthanized two hours after administration of the MCR400, the luminescence spectrum of the tumor location that received the MCR400 indicates the presence of two interesting peaks: the first one at 569 nm that is not present in the spectra of the tumor zones and healthy zones not containing the MCR400 and the second one at 576 nm. We attribute these two peaks to the emission of MCR400 at 569 nm and to that of rhodamine B and lipo-pigments at 576 nm. The peak at 576 nm can not only come from the luminescence of lipo-pigments, because its relative luminescence intensity, I₅₇₆/I₅₀₀˜1, is higher than that of I₅₇₆/I₅₀₀˜0.5 measured in the luminescence spectra of the healthy and tumor locations not containing MCR400, where I₅₇₆ and I₅₀₀ are the luminescence intensities measured at 576 nm and 500 nm respectively.

For the rat 4 euthanized four days after administration of the MCR400, the luminescence spectrum of the tumor location that received the MCR400 indicates the presence of the rhodamine B and lipo-pigments peak at 576 nm. The MCR400 peak at 569 nm is no longer present. This could be explained by the dissociation of rhodamine B from the MCR400, following the administration of MCR400 in the rat brain.

In order to study if this dissociation is due to a variation of pH, we first study whether the MCR400 internalize in viable cells, which could result in a pH variation. MCR400 and 500,000 MDA-MB-231 cells are placed in the presence of a cell viability probe, calcein-acetoxy-methyl ester (Ca), which is not naturally luminescent and which luminesces in the presence of living cells. The cells are incubated for 6 hours either in the presence of Ca or in the presence of Ca and MCR400 where the iron oxide concentration of the MCR400 suspensions is 31.5 μg/ml. The flow cytometer enables to detect either the luminescence of Ca (FL1-H signal), or that of rhodamine B (FL3-H signal). The FL1-H signal is weak at 2-3 for single cells and increases to 100 when cells are in the presence of Ca indicating the viability of MDA-MB-231 cells in the absence of MCR400. When the cells are incubated in the presence of Ca and MCR400, the FL1-H signal is larger than ˜10 and the majority of the cells are therefore viable. As for the FL3-H signal, it indicates that 99% of the cells are luminescent in the presence of MCR400. In order to study MCR400 internalization, MCR400 at a concentration of 100 μg/ml in iron oxide are placed in the presence of U87-Luc cells and macrophages in their respective DMEM and RPMI culture medium with 10% of de-complemented fetal calf serum at 37° C. for 2 hours. The U87-Luc cells were washed five times with PBS (phosphate buffer saline) and fixed, included in resin, and imaged by TEM (transmission electron microscopy). The macrophages were washed five times with PBS and then imaged by epi-fluorescence optical microscopy. In both cases, internalization of MCR400 in lysosomes was observed. During the internalization of magnetosomes in lysosomes, the pH would acidify to a value typically between 3.5 and 5, which could lead to the dissociation of rhodamine B from magnetosomes.

A suspension of MCR400 at 400 μg/mL in iron oxide and a 125 μM solution of rhodamine B were mixed with solutions of hydrochloric acid or with solutions of sodium hydroxide at concentrations ranging from 0.1 to 12 M to adjust the pH to values between 2 and 12. The samples containing the MCR400 suspensions thus treated are then placed against a magnet of 0.6 T for 12 hours at 4° C. and the supernate of the treated MCR400 suspensions is removed. The maximum luminescence intensity of the supernate of the MCR400 suspension, excited at 550 nm, decreases from 140 a.u. at pH 2 down to 20 a.u. at pH 12. On the other hand, the maximum luminescence intensity of rhodamine B alone, excited at 550 nm, increases from 440 a.u. up to 580 a.u. This difference in behavior suggests that rhodamine B associated with MCR400 undergoes during a pH variation a transformation such as a dissociation or chemical modification, which is different from that of rhodamine B. Moreover, the emission wavelength of MCR400 lies between 568 nm and 576 nm for pH>4, values lower than the emission wavelengths of rhodamine B alone and the supernate of MCR400, lying between 577 nm and 580 nm for pH>4. In contrast, for 2<pH<4, the emission wavelengths of rhodamine B alone, MCR400 and the supernate of MCR400, are similar, lying between 580 nm and 584 nm. These behaviors could be explained by the association of rhodamine B with magnetosomes for pH>4 and the dissociation of rhodamine B from magnetosomes for pH<4.

We can conclude that:

(i) It is possible to detect luminescence variation of MCR400 within rat tumors. This luminescence variation could be attributed to a pH variation induced by the internalization of MCR400 in cells, particularly within organelles at acidic pH, such as lysosomes or endosomes, as we observed by electron transmission microscopy.
(ii) This MCR400 luminescence variation as a function of pH could be attributed to the dissociation of rhodamine B from magnetosomes at acidic pH.

### Example 7: Use of MCR400, BNF-Dil and MC-Dil as Temperature and Magnetic Field Probe

### Material and Methods:

For experiments carried out on mouse brains, MCR400 are introduced into brains extracted from mice. In this case, 2 μl or 20 μl of a suspension of MCR400 at a concentration of 20 mg/ml in maghemite are introduced at a depth of 1 mm in the brain.

For experiments in crushed tissue, MCR400 magnetosomes are mixed with previously crushed brain tissue. In this case, 2 μl or 20 μl of a suspension of MCR400 at concentration 20 mg/ml in maghemite are mixed with brain tissue contained in a volume of 2 mm³.

The fluorescence of the MCR400 suspension introduced into the mouse brain or mixed with the tissue is then excited at 405 nm using a laser diode and is detected by a fibered luminescence spectrometer described in Example 6. The optical fiber is positioned at a distance of 3 mm above the surface of the brain or crushed tissue. This distance has been optimized to allow the detection of the maximum fluorescence intensity emitted by MCR400. The fluorescence of MCR400 is measured either in the absence of application of an alternating magnetic field or in the presence of an alternating magnetic field of frequency 198 kHz and strength 8 mT or 25 mT. The application of the alternating magnetic field produces a magnetic excitation of the MCR400. The temperature of the region where MCR400 are located is also measured using an Easir 2 thermographic infra-red camera and a microprobe thermocouple (IT-18, Physitemp, Clifton, USA).

For the experiments carried out in water, 300 μl of three suspensions containing either MCR400, MC-DiI or BNF-Dil at a concentration of 3.6 mg/mL in maghemite are placed inside a tube and exposed to the application of an alternating magnetic field of strength 25 mT and frequency 198 kHz for different durations of 0, 100, 200, 300, 600, 900 or 1200 seconds. The luminescence of these suspensions is measured for these suspensions mixed with their supernate, the supernate of these suspensions and these suspensions mixed in water. The suspensions mixed in their supernate are the suspensions obtained after application of the alternating magnetic field. The supernate of these suspensions is obtained by positioning a 0.6 T Neodinium magnet against the quartz tube containing these suspensions to maintain the different nanoparticles stuck to the wall of the tube. The supernatant is then aspirated using a pipette. The suspensions mixed in water are obtained by removing the supernate from the suspensions and replacing it with water. The luminescence of these three sample types is excited at 405 nm for MCR400 and 550 nm for BNF-DiI and MC-DiI.

### Results and Discussion:

When 2 μl (40 μg) or 20 μl (400 μg) of a suspension of MCR400 is introduced into the brain of a mouse without the application of a magnetic field, we have shown that neither the luminescence intensity nor the temperature of the MCR400 varies over time. A similar result is obtained when MCR400 are exposed to the application of an alternating magnetic field of frequency 198 kHz and intensity 8 mT.

On the other hand, when 2 μl of a suspension of MCR400 are introduced into the brain of a mouse and the MCR400 are exposed a first time (passage 1) to the application of an alternating magnetic field of frequency 198 kHz and strength 25 mT, the luminescence intensity and the temperature of the MCR400 increase from 5 arbitrary units (a.u.) to 30 a.u., i.e. 80%, and from 13° C. to 14° C., respectively, during the first 100 seconds of the application of the field. After a first magnetic treatment of 30 minutes (passage 1), MCR400 are exposed to 4 other successive magnetic excitations (passages 2 to 5). For the passages 2 to 5, the variations of luminescence are always present but less pronounced than for the passage 1. Variations of temperature are no longer observed.

When 20 μl of a suspension of MCR400 are introduced in the brain of a mouse and the MCR400 are exposed for 30 minutes to the application of an alternating magnetic field of frequency 198 kHz and average strength 25 mT, the luminescence intensity and temperature increase. During the first 100 seconds of the magnetic excitation, the luminescence intensity increases from 20 a.u. to 90 a.u., i.e. by ˜80% for 20 μl of administered MCR400. The percentage of luminescence increase, ˜80%, is similar for 2 μl and 20 μl of administered MCR400. During the first 100 seconds of the magnetic excitation, the temperature of the MCR400 increases from 9 to 12° C. The percentage of temperature increase of ˜33% is significantly lower than the percentage of luminescence increase.

When 2 μl of a suspension of MCR400 are mixed with brain tissue and the MCR400 are exposed a first time during thirty minutes to the application of an alternating magnetic field of frequency 198 kHz and strength 25 mT, the luminescence and temperature of MCR400 increase during the first 100 seconds of magnetic excitation by a factor of 30 and 2° C., respectively, two larger increases than those observed in the brain.

When 300 μl of suspensions containing 3.6 mg/ml in maghemite of MCR-400, BNF-Dil and MC-Dil are exposed to the application of a magnetic field of strength 25 mT and frequency 198 kHz during different times of 0, 100, 200, 300, 600, 900 and 1200 seconds, a strong increase in the luminescence of the supernate is observed during the first 100 seconds of the magnetic excitation, from 1 a.u. to 19 a.u. for the supernate of MCR400, from 0 a.u. to 0.32 a.u. for the supernate of BNF-Dil and from 0 a.u. to 0.2 a.u. for the supernate of MC-Dil, then the luminescence is relatively stable after 100 seconds at 18.5-19.5 a.u. for the supernate of MCR-400, at 0.31-0.33 a.u. for that of the BNF-Dil and at 0.17-0.2 a.u. for that of MC-Dil. In contrast to the luminescence of the supernate which does not contain particles, the luminescence of MCR-400, BNF-Dil and MC-Dil mixed with their supernate or water remains stable at a low value of ˜4 for MCR400 or remains at zero for BNF-Dil and MC-Dil when the alternating magnetic field of strength 25 mT and frequency 198 kHz is applied for different durations of 0, 100, 200, 300, 600, 900 and 1200 seconds.

We also measured the slope at the origin of the luminescence variation of MCR400 introduced into mouse brains or mixed with tissue, ΔF/δt, as a function of the slope at the origin of the temperature variation, ΔT/δt. As ΔT/δt increases, ΔF/δt increases and a relation close to a linear relation, can be established between ΔF/δt and ΔT/δt with a coefficient (ΔF/δt)/(ΔT/δt)˜45 a.u./° C. Moreover, when ΔT/δt˜0, a nonzero fluorescence variation can be detected, ΔF/δt˜0.03.

We can conclude from this example that:

(i) when the strength of the alternating magnetic field is lower than a certain threshold (B<8 mT), it produces neither a luminescence variation nor a temperature variation of the MCR400. The magnetic excitation of the MCR400 resulting in a variation of luminescence and/or of temperature requires the application of an alternating magnetic field of strength larger than 8 mT.
(ii) when MCR400 are introduced in a brain or mixed with tissue and exposed to an alternating magnetic field of strength larger than ˜25 mT, this may induce an increase in luminescence and temperature. The variation of the luminescence is more pronounced than that of the temperature. Moreover, the luminescence variation can be detected under conditions where the temperature variation can't be detected, especially at low concentrations in iron oxide nanoparticles. This makes this luminescent probe a more sensitive local probe than a macroscopic temperature probe that measures an average temperature over a larger volume.
(iii) The increased luminescence of MCR400 induced by the application of an alternating magnetic field could be associated with the dissociation of rhodamine B from magnetosomes. Luminescence would initially be quenched when rhodamine B is associated with iron oxide nanoparticles and would increase when rhodamine B dissociates from MCR400, following the application of the alternating magnetic field. Then, rhodamine B would diffuse into the probe environment, which could explain the sometimes random variations in luminescence intensity observed after 100 seconds.
(iv) The increase in luminescence of the supernate of MCR400 could be due to an increase in temperature caused by the magnetic field. In contrast, the increase in luminescence of the supernate of BNF-DiI is not due to the temperature increase induced by the magnetic field, but to a non-thermal effect induced by the magnetic field.
(v) After having exposed MCR400 to an alternating magnetic field of strength 25 mT and frequency 198 kHz two times for more than 30 minutes, the increase in luminescence of MCR400 produced by the application of the alternating magnetic field decreases. This quenching could be due to the diffusion of MCR400 under the alternating magnetic field or the decrease in the number of rhodamine B molecules associated with magnetosomes.
(vi) the luminescence and temperature variations of the MCR400 induced by the application of an alternating magnetic field of strength 25 mT and frequency 198 kHz are correlated. The more the luminescence variation increases, the more the temperature variation increases. Moreover, a quasi-linear relationship between ΔF/δt and ΔT/δt can be established for a series of different conditions: (i), for different quantities of iron oxide nanoparticles tested (40 μg or 400 μg), (ii), for different types of nanoparticles (magnetosomes and BNF), (iii), for different types of fluorophores (Rhodamine B and Dil) and for different types of preparations (MCR400 mixed with tissue, inserted into the brain or mixed in water).
(vii) In order to establish the relationship between luminescence variation and temperature variation, it is possible to consider the variations occurring during the first 100 to 200 seconds of excitation due to the alternating magnetic field. Indeed, after this initial period of 100 to 200 seconds, the luminescence variation of the MCR400 can be random.
(viii) when a suspension of MCR400, MC-Dil, BNF-Dil is exposed to the application of an alternating magnetic field of strength 25 mT and frequency 198 kHz, this produces an increase in the luminescence intensity of the supernate observed during the first 100 seconds of magnetic excitation. This increase in luminescence could be explained by the dissociation of rhodamine B or Dil from magnetosomes or BNF. When Rhodamine B or Dil is free in solution and the iron oxide nanoparticles have been removed, their luminescence would increase because it would no longer be quenched by iron oxide nanoparticles.
(ix) The luminescence of the MCR400, BNF-Dil and MC-Dil mixed with the supernate or with water does not vary after application of an alternating magnetic field of strength 25 mT and frequency 198 kHz. This behavior could be explained by the absorption of light by the iron oxide nanoparticles.

### Example 8: Measurement of the Life Time of Rhodamine B and MCR400

Life time experiments were carried out using the frequency method. The set-up uses a modulated laser diode as excitation source, a confocal fluorescence spectroscope, a control unit for intensifying images and a camera, frequency generators and a spectrograph controller. The modulation frequency has been varied between 30 and 180 MHz. The excitation wavelength is 445 nm. The laser light is modulated by a radio frequency signal. An interference filter suppresses parasitic fluorescence from the laser diode and a high pass filter is placed in the detection path to decrease scattering. A volume of 1 mL of a suspension of MCR400 at 5 mg/mL in iron oxide and rhodamine B at a concentration of 5 μM was placed in a dry heating bath for 5 minutes to heat the suspensions at 25° C. and at 45° C. 10 μl of a suspension of MCR400 at 5 mg/ml in iron oxide and rhodamine B at a concentration of 5 μM were deposited on a slide and then placed in front of an objective ×40 to measure the fluorescence lifetime of each suspension. At 25° C., the fluorescence lifetime is 4.8 ns for the MCR400 and 1.6 ns for rhodamine B. When the samples are heated to 45° C., the life time of MCR400 is 1.6 ns and that of rhodamine B is 1.3 ns.

We can conclude from this example that:

(i) The fluorescence lifetime of rhodamine B is shorter than that of MCR400 at 25° C. and 45° C.
(ii) Rhodamine B is well associated with magnetosomes in MCR400 because the fluorescence lifetime of Rhodamine B is shorter than that of MCR400.
(iii) The life time of MCR400 decreases more significantly when the temperature is increased than that of Rhodamine B.
(iv) The life time of the MCR400 at 45° C. is identical to that of rhodamine B alone, indicating the dissociation of rhodamine B from magnetosomes.

### Example 9: Use of NANOF Associated to a Pharmaceutical Compound

The pharmaceutical compound is associated with NANOF by a chemical reaction when the compound is mixed with NANOF. The particle is then sent to or inserted into an organism or part of an organism, such as a tumor or a tumor cell. The particle is then exposed to a magnetic field, such as an alternating magnetic field, or to a modification in pH, temperature, or chemical composition of the medium surrounding the particle, which induces the dissociation of the pharmaceutical compound from the NANOF and/or chemical modification of NANOF. Following this dissociation, the compound is activated and acts as a diagnostic or therapeutic treatment tool.

### Example 10: Synthesis of Pyrogen-Free and Luminescent NANOF, Synthesized by Magnetotactic Bacteria, Covered with a Coating of Poly-1-Lysine and Dil (M-PLL-DiI)

The non-pyrogenic and luminescent NANOF suspensions of M-PLL-DiI are prepared under a hood in pyrogen-free conditions using the central parts of the MSR-1 magnetosomes which are prepared as follows. MSR-1 bacteria are first cultivated at 30° C. for 5 to 7 days on an agar gel in the presence of iron and at low oxygen concentration (˜0.5% 02). Magnetic colonies are removed and cultivated at 30° C. in the presence of air for several days in an iron-free pre-culture medium containing carbonaceous, nitrogen, mineral, trace element and yeast extract sources. Magnetotactic bacteria collected from pre-culture are cultivated in a 50 liter fermenter at 30° C. in a medium similar to the pre-culture medium. During growth, the pH is maintained at 6.8-7 by adding an acidic nutriment medium containing an iron source and compressed air is introduced into the culture medium to promote bacterial growth while maintaining oxygen concentration below 0.2% to allow the synthesis of magnetosomes. The MSR-1 bacteria originating from the fermentation are concentrated to an optical density, measured at 565 nm (OD₅₆₅), of 110-120. 100 ml of this bacterial concentrate are then mixed with 400 ml of 5M NaOH and heated at 60° C. for 1.5 hours to 2 hours to lyse the bacteria. The treated magnetosomes are then isolated from the bacterial debris by placing a Neodinium magnet overnight against the wall of the container containing the lysed bacteria suspension and by replacing the supernate containing the NaOH and bacterial debris with 1×PBS. The resulting suspension is then sonicated for 20 seconds at 10 W in the presence of 1×PBS, placed against a Neodinium magnet for 15 minutes, the supernate is removed and the treated magnetosomes are resuspended in 1×PBS. This sequence of sonication and magnetic separation is repeated four times. Then, 100 mL of the resulting suspension is mixed with 200 mL of a solution containing 1% Triton X-100 and 1% SDS, the mixture is heated overnight at 50° C., is placed against a magnet of Neodinium, the supernate is removed and replaced by 80 mL of phenol at pH 8. The obtained suspension is heated for 2 hours by sonication at 60° C., maintained overnight at 60° C. without sonication, placed against a magnet, the supernate of the suspension is removed and replaced by 80 mL of chloroform. The suspension containing the chloroform is placed against a Neodinium magnet, the supernate is removed and the residual chloroform adsorbed at the surface of the treated magnetosomes is removed by heating these magnetosomes (without supernate) for 2 hours in a hood. Finally, the central parts of the magnetosomes obtained are desorbed from the glass wall of the tubes containing them by adding 80 ml of 1M NaOH heated for 1 hour at 60° C. in an ultrasonic bath. The suspension containing the central parts of the magnetosomes is placed against a Neodinium magnet, the supernate is removed and replaced with sterile MilliQ water, the suspension is sonicated for 20 seconds at 10 W. This washing sequence is repeated four times. The suspension containing the central parts of the magnetosomes is degassed with nitrogen to avoid oxidation, sterilized by autoclaving and stored at −80° C.

The suspensions containing the central parts of the magnetosomes are characterized by an endotoxin concentration between 10 and 100 EU/mg/ml, a low stability and rapid sedimentation, the absence of a coating as observed by transmission electron microscopy. 10 ml of a suspension containing 56 mg of the central parts of the magnetosomes are mixed with 373 mg of poly-L-lysine hydrobromide of molar mass 21000 g/mol (Gmac, CAS: 25988-63-0) and 250 μg of Dil. The mixture is sonicated in an ultrasonic bath at 25 kHz at 30° C. for 15 minutes. Then the pyrogen-free mixture is stirred at 4° C. on a wheel at a speed of 13 rotations per minute for 19 hours. Then the mixture is sonicated with a sonicating finger for 10 minutes at 10 W with pulses of one minute and an interval of one minute between pulses, protected from light and at 5° C. The suspension is concentrated by centrifugation at 14,000 rpm for 30 minutes at 5° C. and then washed twice with 0.5% ethanol and fifteen times with sterile Millipore® water. The suspension thus obtained is finally resuspended in sterile Millipore® water to obtain a suspension of pyrogen-free M-PLL-DiI.

The 3T3 cells are seeded in Petri dishes (ø 30 mm) at a concentration of 5·10⁵ cells per 2 ml in a culture medium (DMEM) containing 10% of NCBS. After 24 hours, they are rinsed with PBS and then treated for 24 hours with a suspension of M-PLL-DiI (250 μg/ml in iron). After treatment, the cells are rinsed three times with culture medium with 10% NCBS and the cells are then photographed using an epi-fluorescence microscope. Cellular internalization of MPLL-DiI is observed by fluorescence.

### Examples 11: Properties of MCR400 Compared with Those of MC

Electron transmission microscopy measurements reveal that MCR400 have an average size of 60 nm and a size distribution between 15 nm and 90 nm, while MC have an average size of 40 nm and a size distribution between 5 nm and 60 nm. Due to the increase in magnetosome size in MCR400 by 50% compared with MC, we can expect an increase in coercivity, ratio between remanent and saturation magnetization or SAR of at least 0.01%, 0.1%, 1%, 2%, 5%, 10%, 25%, or 50% in MCR400 compared with MC.

1. A particle probe comprising:
at least one ferrimagnetic or ferromagnetic iron oxide nanoparticle associated with at least one compound, wherein the at least one ferrimagnetic or ferromagnetic iron oxide nanoparticle is not bound to the at least one compound by at least one nucleic acid or at least one amino acid, in which the at least one compound dissociates from the at least one ferrimagnetic or ferromagnetic iron oxide nanoparticle and/or chemically modifies itself, where the dissociation and/or chemical modification of the at least one compound does not lead to its destruction following a physicochemical disturbance applied on the particle probe, where the physicochemical disturbance induces modification in the condition of the at least one ferrimagnetic or ferromagnetic iron oxide nanoparticle.

2. The particle probe according to claim 1, in which the at least one ferrimagnetic or ferromagnetic iron oxide nanoparticle is synthesized by a magnetotactic bacterium and does not comprise any carbonaceous material originating from the bacterium.

3. The particle probe according to claim 1, wherein the at least one compound is associated with the at least one ferrimagnetic or ferromagnetic iron oxide nanoparticle by weak or strong bonds.

4. The particle probe according to claim 1, wherein the at least one compound is a luminescent substance.

5. The particle probe according to claim 1, wherein the at least one compound is a luminescent substance having a luminescence intensity associated with the at least one ferrimagnetic or ferromagnetic iron oxide nanoparticle that is quenched by the at least one ferrimagnetic or ferromagnetic iron oxide nanoparticle compared with the luminescence intensity of the luminescent substance alone.

6. The particle probe according to claim 1, wherein the at least one compound is a luminescent substance, and dissociation of the luminescent substance from the at least one ferrimagnetic or ferromagnetic iron oxide nanoparticle and/or chemical modification of the luminescent substance is related to a luminescence variation of the luminescent substance.

7. The particle probe according to claim 1, wherein the at least one compound is a luminescent substance and a luminescence variation of the luminescent substance associated with the at least one ferrimagnetic or ferromagnetic iron oxide nanoparticle is different from that of the luminescent substance alone.

8. The particle probe according to claim 1, wherein the at least one compound is a luminescent substance which is a luminophore, a chromophore or a fluorophore.

9. The particle probe according to claim 1, wherein the at least one compound is a luminescent substance, and the luminescence substance has at least one chemical function selected from the group consisting of carboxylic acid, phosphoric, sulfonic acid, ester, amide, ketone, alcohol, phenol, thiol functional groups, amine, ether, sulfide, acid anhydride, acyl halide, amidine, nitrile, hydroperoxide, imine, aldehyde, peroxide, and an acid, basic, oxidized, reduced, neutral, or positively or negatively charged derivative of these functions.

10. The particle probe according to claim 1, wherein the physicochemical disturbance is applied on the particle by radiation.

11. The particle probe according to claim 1, wherein the physicochemical disturbance applied on the particle is due to a variation of the environment of the particle.

12. A method for detecting luminescence variation, comprising a step of detecting luminescence variation of the particle probe as defined in claim 1.

13-17. (canceled)

18. A medical device comprising at least one particle probe as defined in claim 1.

19-20. (canceled)
